(19) United States
(12) Patent Application Publication
(10) Pub. No.: US 2022/0054616 A1
(43) Pub. Date: Feb. 24, 2022

(54) Attenuated Bordetella Bronchiseptica Strains, Oral Vaccines Containing the Attenuated Strains, and Methods of Making & Use Thereof

(71) Applicant: BOEHRINGER INGELHEIM ANIMAL HEALTH USA INC., DULUTH, GA (US)

(72) Inventors: Laurent Bernard FISCHER, Sainte Foy Les Lyon (FR); Edmond JOLIVET, Orlienas (FR); Kevin MILLSAP, Bogart, GA (US)

(21) Appl. No.: 17/417,327

(22) PCT Filed: Jan. 6, 2020
(86) PCT No.: PCT/US20/12406

§ 371 Date: Jun. 22, 2021

Related U.S. Application Data
(60) Provisional application No. 62/788,764, filed on Jan. 4, 2019.

(51) Int. Cl. A61K 39/02; C12N 7/00

(52) U.S. Cl. CPC A61K 39/099; A61K 2039/522; C12N 7/00

(57) ABSTRACT

The present invention provides attenuated, aroA mutant B. bronchiseptica strains that are effective to elicit an immune response in an animal against B. bronchiseptica. Also provided are immunogenic compositions and vaccines which include the attenuated, aroA mutant B. bronchiseptica strains. Also provided are kits for use with such compositions and vaccines. Also provided are methods of orally administering attenuated, aroA mutant B. bronchiseptica strains, compositions, and vaccines to animals.

### CROSS-REFERENCE TO RELATED APPLICATIONS

This application claims priority to U.S. provisional application 62/788,764 filed on Jan. 4, 2019, the entire contents of which are hereby incorporated by reference herein.

### INCORPORATION BY REFERENCE

All references cited herein are incorporated by reference herein in their entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is MER 17-336_ST25 (sequence listing).txt. The text file is 22 KB; it was created on 20 Dec. 2019; and it is being submitted electronically via EFS-Web, concurrent with the filing of the specification.

### FIELD OF THE INVENTION

The present invention relates generally to Bordetella bronchiseptica bacterial strains, compositions, and vaccines, and methods of manufacture and use thereof.

### BACKGROUND

In veterinary medicine, B. bronchiseptica leads to a range of host-determined pathologies, and it causes serious disease in canines, porcines, and rabbits. In porcines, B. bronchiseptica and P. multocida combine to cause atrophic rhinitis. In canines, B. bronchiseptica causes acute tracheobronchitis, typified by a harsh, honking “Kennel” cough. Such a cough may also be caused by canine adenovirus-2 (cAV2) and/or canine parainfluenza virus (cPI2). In felines, B. bronchiseptica infection is associated with tracheobronchitis, conjunctivitis, and rhinitis (as called “upper respiratory tract infection”, or “URI”), mandibular lymphadenopathy, and pneumonia. However, feline URI can also be caused by herpesvirus, calicivirus, Mycoplasma spp., and/or Chlamydia psittaci.

B. bronchiseptica is well known for high frequency phase variation and antigenic modulation (Monack, D. M., et al., “Phase Variants of Bordetella bronchiseptica Arise by Spontaneous Deletions in the Vir Locus.” Molecular Microbiology, vol. 3, no. 12, 1989, pp. 1719-1728). Therefore, from one strain to another, and depending on culture conditions, the expression level of antigenic determinants can vary and impact the immunogenicity of inactivated vaccine preparations. Classically, B. bronchiseptica strains (e.g. RB50) grown at 37° C. produce proteins that are involved in virulence and known to be important antigenic determinants, including adenylate cyclase-hemolysin (Ac-Hly), type III secretion system effector (BteA), filamentous hemagglutinin (FHA), pertactin (PRN), and Fimbriae (FIM; encoded by the virulence activated genes named vag). However, the bacterium represses production of these virulence factors when it is grown at 25° C. or at 37° C. in presence of MgSO₄ or Nicotinic acid. Further, expression of virulence repressed genes named vrg such as those coding flagellum is activated in these conditions.

Intranasal vaccination has been generally regarded as the only acceptable method in the art for vaccinating animals against B. bronchiseptica. Systemic administration of live B. bronchiseptica vaccines has not been regarded as a safe option since it is known that the systemic administration of live B. bronchiseptica, even when attenuated, can lead to serious abscess formation [see e.g., Toshach et al., J Am Anim Hosp Assoc 33:126-128 (1997)]. Likewise, studies have demonstrated that intranasal vaccination was far superior to oral vaccination (Ellis J. A., et al. “Comparative efficacy of intranasal and oral vaccines against Bordetella bronchiseptica in dogs.” The Veterinary Journal, vol. 212, 2016, pp. 71-77).

A live attenuated/avirulent Bordetella bronchiseptica strain has been shown to provide strong protection against kennel cough in dogs (Bey, R. F., et al., “Intranasal Vaccination of Dogs with Liver Avirulent Bordetella bronchiseptica: Correlation of Serum Agglutination Titer and the Formation of Secretory IgA with Protection Against Experimentally Induced Infectious Tracheobronchitis.” American Journal of Veterinary Research, vol. 42, no. 7, 1981, pp. 1130-1132) when administered intranasally to dogs in a vaccine formulation. A correlation of serum aggutination titer and the formation of secretory IgA with protection against experimentally induced infectious tracheobronchitis was shown after nasal administration. This protection was seen as early as 48 h after vaccination. Intranasal vaccination with live attenuated B. bronchiseptica has also been shown to protect against atrophic rhinitis in two-days old piglets (De Jong, M. F. “Prevention of Atrophic Rhinitis in Piglets by Means of Intranasal Administration of a Live Non-AR-PathogenicBordetella Bronchisepticavaccine.” Veterinary Quarterly, vol. 9, no. 2, 1987, pp. 123-133). Prevention of atrophic rhinitis in piglets by means of intranasal administration of a live non-AR-pathogenic Bordetella bronchiseptica vaccine indicates that, in a live attenuated form, Bordetella vaccines can be active in new-born animals.

An aroA deletant strain of B. bronchiseptica also has been constructed and employed solely in an intranasal vaccine (Stevenson and Roberts, Vaccine 20, 2325-2335 (2002)). However, inactivation of the aroA gene highly attenuates B. bronchiseptica, severely impairing its ability to colonize and survive in the respiratory tract. This is consistent with similar studies wherein an aroA-deleted B. pertussis was highly attenuated, but it had also lost its capacity to colonize the respiratory tract of the intranasally vaccinated animals, and induced protective immunity only after repeated administrations of high doses (Roberts, et al. “Construction and Characterization in Vivo of Bordetella Pertussis AroA Mutants.” Infection and Immunity, American Society for Microbiology Journals, 1 Mar. 1990, iai.asm.org/content/58/3/732.short.).

Intranasal vaccines are inconvenient to administer, especially to animals that often resist administration of any substance into their nostrils, such as canines or felines. Administering vaccines intransally also creates a risk that the amount of vaccine taken in by the animal will be significantly less than the dose shown to be protective, should the animal sneeze during the administration.

In addition to the above limitations of existing B. bronchiseptica vaccines and methods, efforts to provide multivalent vaccines using existing B. bronchiseptica strains combined with other bacterial and viral strains have had limited success. For example, Skibinski et al., described numerous challenges associated with developing combination vaccines, including a reduced response to one of the antigens. Skibinski, David A G, et al., “Combination Vaccines.” Journal of Global Infectious Diseases, vol. 3, no. 1, 2011, p. 63., doi:10.4103/0974-777x.77298.

Existing B. bronchiseptica vaccines suffer from other limitations as well, such as unwanted side effects from vaccine excipients essential to the existing vaccine formulations. Adjuvants used to boost vaccine efficacy increase local reactions (such as redness, swelling, and pain at the injection site) and systemic reactions (such as fever, chills and body aches) (“Vaccine Safety.” Centers for Disease Control and Prevention, Centers for Disease Control and Prevention, 22 Oct. 2018, www.cdc.gov/vaccinesafety/concerns/adjuvants.html.) Serum used in existing vaccines is rich in proteins and increases risk of allergic reactions. For example, fetal calf serum (FCS) is a typical substance of animal origin (SAO) which can cause an allergic reaction (Ohmori, Keitaro, et al. “IgE Reactivity to Vaccine Components in Dogs That Developed Immediate-Type Allergic Reactions after Vaccination.” Veterinary Immunology and Immunopathology, vol. 104, no. 3-4, 2005, pp. 249-256., doi:10.1016/j.vetimm.2004.12.003).

Therefore, what is needed are improved B. bronchiseptica vaccines that are safe, effective, and suitable for non-intranasal delivery.

### SUMMARY OF THE INVENTION

Provided herein are attenuated aroA mutant B. bronchiseptica strains capable of eliciting protective immunity against B. bronchiseptica infection in an animal when administered orally to the animal. In embodiments, the attenuated aroA mutant B. bronchiseptica strain has a partially deleted aroA gene. In embodiments, the attenuated aroA mutant B. bronchiseptica strain has a complete deletion of its aroA gene. In embodiments, the attenuated aroA mutant B. bronchiseptica strain comprises a polynucleotide having at least 85% sequence identity to SEQ ID NO:3. In embodiments, the attenuated aroA mutant B. bronchiseptica strain is deposited under the CNCM Deposit No. 1-5391.

Also provided herein are immunogenic compositions comprising an attenuated aroA mutant B. bronchiseptica strain capable of eliciting an immune response when administered orally to an animal. In embodiments, the immunogenic composition further comprises a pharmaceutically or veterinarily acceptable carrier, adjuvant, vehicle, and/or excipient. In embodiments, the immunogenic composition is adjuvant-free. In embodiments, the immunogenic composition is a single dose formulation for oral administration. In embodiments, the single dose formulation has between 1×10³ CFU to 1×10¹⁰ CFU of the attenuated aroA mutant B. bronchiseptica strain. In embodiments, the single dose formulation has between 1×10⁸ CFU to 1×10¹⁰ CFU of the attenuated aroA mutant B. bronchiseptica strain. In embodiments, the immunogenic composition further comprises a canine parainfluenza virus antigen. In embodiments, the immunogenic composition further comprises a canine adenovirus antigen. In embodiments, the immunogenic composition is free of substances of animal origin. In embodiments, the immunogenic composition is a vaccine.

Also provided herein are methods for eliciting a protective immune response against B. bronchiseptica in an animal, comprising administering to the animal an oral vaccine comprising an effective amount of an aroA mutant Bordetella bronchiseptica bacteria strain. In embodiments, the animal is a canine or a feline. In embodiments, the protective immune response is effective to provide the animal with protection against virulent B. bronchiseptica infection, clincical disease associated with virulent B. bronchiseptica infection, and/or clinical symptoms associated with virulent B. bronchiseptica infection. In embodiments, the method employs a prime-boost administration regimen. In embodiments, the animal is between 0 to 6 months old.

### BRIEF DESCRIPTION OF DRAWINGS

FIG. 1 shows a map of DNA plasmid pBP1070.

FIG. 2 is a flow diagram schematizing the integration of the pPB1070 suicide plasmid into the chromosome of the B. bronchiseptica strain 05.

FIG. 3 is a flow diagram schematizing the second step for producing a mutant B. bronchiseptica strain. Here, suicide plasmid pPB1070 excises aroA from the chromosome of B. bronchiseptica strain 05, producing a mutant ΔaroA version of strain 05.

FIGS. 4A-4D show the results of the functional characterization in E. coli of the pPB1070 suicide plasmid for the aroA deletion in B. bronchiseptica. FIG. 4A shows the growth on LB/Kanamycin plates; suicide plasmid with sacB & clones 4 & 5. FIG. 4B shows the growth on LB/sucrose (2.5%) plates; suicide plasmid with sacB & clones 4 & 5. FIG. 4C shows the growth on LB/kanamycin plates; clones 1 to 3. FIG. 4D shows the growth on LB/sucrose (2.5%) plates; clones 1 to 3. As indicated by the results, sacB has been placed under the control of the porine promoter.

FIGS. 5A-5C show the functional characterization of the integrated pPB1070 suicide plasmid for aroA deletion in B. bronchiseptica. Serial dilution and dots of each clone culture on BG plates: kanamycin (FIG. 5A), Sucrose 5% (FIG. 5B), and Sucrose 10% (FIG. 5C). FIG. 5A shows bacterial growth on BG plates+kanamycin. FIG. 5B shows bacterial growth on 5% sucrose. FIG. 5C shows bacterial growth on 10% sucrose. The counterselection sacB/Sucrose appears functional in Bb.

FIGS. 6A-6C show additional functional characterization of the integrated pPB1070 Suicide plasmid for aroA deletion in B. bronchiseptica. Serial dilution and dots of each clone culture on BG plates: kanamycin (FIG. 6A), Sucrose 5% (FIG. 6B) and Sucrose10% (FIG. 6C). FIG. 6A shows bacterial growth on BG+Kanamycin. FIG. 6B shows bacterial growth on BG+5% sucrose. FIG. 6C shows bacterial growth on BG+10% sucrose. The counterselection sacB/Sucrose appears functional in Bb at 48H.

FIGS. 7A-7B show the results of ΔaroA deletion mutant screening using the sacB/sucrose counter-selection system. The plate in FIG. 7A has 5% sucrose. FIG. 7B is a replica plate of the sucrose plate shown in FIG. 7A, containing kanamycin.

FIG. 8A-C show the identification of thirteen new H+ (Hemolytic clone) aroA-gene deleted B. bronchiseptica mutants using various agar plates. The plate in FIG. 8A has (BG+5% Blood, top; the plate in FIG. 8B has BG+5% blood+1× aromix, middle; the plate in FIG. 8C has BG+5% blood+1× aromix+1× kanamycin).

FIG. 9 is a gel showing the identification of an H+(Hemolytic clone) aroA-gene deleted mutant in Bordetella bronchiseptica.

FIG. 10 shows the PCR results demonstrating the mutant status of the thirteen delta aroA B. bronchiseptica clones.

FIG. 11 is a graph showing the clinical signs in canines post administration of the indicated treatments, followed by virulent challenge with virulent B. bronchiseptica.

FIG. 12 is a graph showing the B. bronchiseptica Global Clinical signs for groups A and C.

FIG. 13 shows results on clinical scores for B. bronchiseptica ΔaroA cultured in different media.

FIG. 14 shows a table detailing the sequence listing.

### DETAILED DESCRIPTION OF THE INVENTION

The present disclosure provides mutant Bordetella bronchiseptica bacteria having a mutated or deleted aroA gene such that a protein crucial to the production of aromatic amino acids encoded by the aroA gene is either non-functional when expressed or not produced at all. The mutant Bordetella bronchiseptica bacteria can be made, starting with a highly virulent parent strain (e.g., a wild-type strain), by engineering the parent strain's aroA gene to have a mutation or deletion of one or more nucleotides. Surprisingly, the mutant Bordetella bronchiseptica bacteria of the present disclosure are attenuated but still retain a high degree of immunogenicity, and are therefore suitable for use in live attenuated immunogenic compositions, live attenuated vaccines, and methods of using the same.

The mutant Bordetella bronchiseptica bacteria, immunogenic compositions, and vaccines of the present disclosure can provide a number of benefits over those existing in the art.

Advantageously, the mutant Bordetella bronchiseptica bacteria strains may not replicate in an animal, and therefore may not shed from the animal. Thus, an animal vaccinated with the mutant Bordetella bronchiseptica bacteria may not shed bacteria.

The mutant Bordetella bronchiseptica bacteria are also able to safely and efficiently elicit a highly protective immune response when delivered via the oral route to an animal. In fact, a single dose oral administration of the mutant Bordetella bronchiseptica strains can be sufficient to confer protective immunity, even in the absence of an adjuvant. These results are surprising because those skilled in the art: (i) believed that B. bronchiseptica colonization/amplification was necessary for the bacterium to elicit a protective immune response, and an aroA mutation/deletion blocks the ability of the B. bronchiseptica bacterium to amplify in vivo, (ii) expected that oral administration of a B. bronchiseptica vaccine would be significantly and prohibitively less efficient than the intranasal route (e.g. oral administration was not a viable/feasible option), and (iii) knew that prior B. bronchiseptica vaccines required adjuvants to be effective.

Another advantage of the mutant Bordetella bronchiseptica bacteria of the present disclosure is that they can be effectively cultured in non-animal Tryptic Soy Broth (TSB-NA). The use of TSB-NA, as opposed to substances of animal origin like animal serums, to culture mutant Bordetella bronchiseptica bacteria that ultimately end up in immunogenic compositions and vaccines can reduce the risk of contamination by adventitious agents, reduce the cost of vaccine components (animal originated materials are more expensive than non-animal), and reduce process variability due to the intrinsic variability in the quality of animal products.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicate otherwise.

As used herein, “animal” includes mammals. The animal may be selected from equine (e.g., horse), canine (e.g., dogs, wolves, foxes, coyotes, jackals), feline (e.g., lions, tigers, domestic cats, wild cats, other big cats, and other felines including cheetahs and lynx), ovine (e.g., sheep), bovine (e.g., cattle), swine (e.g., pig), avian (e.g., chicken, duck, goose, turkey, quail, pheasant, parrot, finches, hawk, crow, ostrich, emu and cassowary), primate (e.g., prosimian, tarsier, monkey, gibbon, ape). The term “animal” also includes an individual animal in all stages of development, including newborn, embryonic, and fetal stages.

As used herein, “antigen” or “immunogen” means a substance that induces a specific immune response in a host animal. The antigen may comprise, for example, a whole organism, killed, attenuated or live; a subunit or portion of an organism; a piece or fragment of DNA capable of inducing an immune response upon presentation to a host animal; and the like

The term “epitope” refers to the site on an antigen or hapten to which specific B cells and/or T cells respond. The term is also used interchangeably with “antigenic determinant” or “antigenic determinant site”. Antibodies that recognize the same epitope can be identified in a simple immunoassay showing the ability of one antibody to block the binding of another antibody to a target antigen.

As used herein, an “aroA mutant” bacterium is a bacterium having a genetic alteration in the aroA gene that results in impairment of the chorismate biosynthetic pathway of the bacterium. An aroA mutant bacterium either cannot synthesize chorismate, or synthesizes significantly less chorismate than a corresponding wild-type bacterium, which consequently leads to a significant inhibition and/or blockage of the growth of the bacterium in an unsupplemented media, environment, or milieu.

As used herein, the term “canine” includes all domestic dogs, Canis lupus familiaris and Canis familiaris, unless otherwise indicated.

As used herein, the term “feline” refers to any member of the Felidae family. Members of this family include wild, zoo, and domestic members, such as any member of the subfamilies Felinae, e.g., cats, lions, tigers, pumas, jaguars, leopards, snow leopards, panthers, North American mountain lions, cheetahs, lynx, bobcats, caracals or any cross breeds thereof. Cats also include domestic cats, pure-bred and/or mongrel companion cats, show cats, laboratory cats, cloned cats, and wild or feral cats.

As used herein, the term “gene” is used broadly to refer to any segment of a polynucleotide associated with a biological function.

An “isolated” biological component (such as a nucleic acid or protein or organelle) refers to a component that has been substantially separated or purified away from other biological components in the cell of the organism in which the component naturally occurs, for instance, other chromosomal and extra-chromosomal DNA and RNA, proteins, and organelles. Nucleic acids and proteins that have been “isolated” include nucleic acids and proteins purified by standard purification methods. The term also embraces nucleic acids and proteins prepared by recombinant technology as well as chemical synthesis.

As used herein a “genetic alteration” or “mutation” of a gene refers to a nucleic acid substitution, deletion, and/or insertion in the gene.

As used herein, the terms “identity” and “sequence identity” refer to a relationship between two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides are identical. The total number of such position identities is then divided by the total number of nucleotides in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988). Preferred methods to determine the sequence identity are designed to give the largest match between the sequences tested. Methods to determine sequence identity are also codified in publicly available computer programs which determine sequence identity between given sequences. In addition to those otherwise mentioned herein, mention is made of the programs BLAST, gapped BLAST, BLASTN, BLASTP, and PSI-BLAST, provided by the National Center for Biotechnology Information. These programs are widely used in the art for this purpose and can align homologous regions of two amino acid sequences.

As used herein, the term “immunogenic composition” refers to a composition that comprises at least one antigen which elicits an immunological response in a host to which the immunogenic composition is administered.

As used herein, an “immunological response” to a composition or vaccine is the development in the host of a cellular and/or antibody-mediated immune response to a composition or vaccine of interest. Usually, an “immunological response” includes but is not limited to one or more of the following effects: the production of antibodies, B cells, helper T cells, and/or cytotoxic T cells, directed specifically to an antigen or antigens included in the composition or vaccine of interest. Preferably, the host will display a therapeutic or protective immunological response such that resistance to new infection will be enhanced and/or the clinical severity of the disease reduced. Such protection can be demonstrated by a reduction or lack of symptoms and/or clinical disease signs normally displayed by an infected host, a quicker recovery time and/or a lowered pathogen titer in the infected host.

As used herein, a “multivalent vaccine” is a vaccine that comprises two or more different antigens. A multivalent vaccine typically can stimulate the immune system of the recipient against two or more different pathogens.

As used herein, the terms “nucleic acid” and “polynucleotide” refer to RNA or DNA that is linear or branched, single or double stranded, or a hybrid thereof. The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracyl, other sugars and linking groups such as fluororibose and thiolate, and nucleotide branches.

As used herein, the terms “pharmaceutically acceptable” and “veterinarily acceptable” are used adjectivally to mean that the modified noun is appropriate for use in a pharmaceutical or veterinary product. When it is used, for example, to describe an excipient in a pharmaceutical or veterinary vaccine, it characterizes the excipient as being compatible with the other ingredients of the composition and not disadvantageously deleterious to the intended recipient.

As used herein, the term “carrier” refers to a diluent, adjuvant, excipient, or vehicle with which the compound is administered. Pharmaceutically acceptable carriers can be sterile liquids, such as water and/or oils, including those of petroleum, animal, vegetable or synthetic origin, such as peanut oil, soybean oil, mineral oil, sesame oil and the like. Water or aqueous solution saline solutions and aqueous dextrose and glycerol solutions can be employed as carriers.

As used herein, an “adjuvant” is a substance that is able to favor or amplify the cascade of immunological events, ultimately leading to a better immunological response, i.e., the integrated bodily response to an antigen. An adjuvant is in general not required for the immunological response to occur, but favors or amplifies this response.

As used herein, the terms “protecting”, “providing protection to”, and “aids in the protection” do not require complete protection from any indication of infection. For example, “aids in the protection” can mean that the protection is sufficient such that, after challenge, symptoms of the underlying infection are at least reduced, and/or that one or more of the underlying cellular, physiological, or biochemical causes or mechanisms causing the symptoms are reduced and/or eliminated. It is understood that reduced, as used in this context, means relative to the state of the infection, including the molecular state of the infection, not just the physiological state of the infection.

As used herein, the terms “protein” and “polypeptide” are used interchangeably herein to refer to polymers of amino acid residues of any length. The polymer can be linear or branched, it may comprise modified amino acids or amino acid analogs, and it may be interrupted by chemical moieties other than amino acids. The terms also encompass an amino acid polymer that has been modified naturally or by intervention; for example disulfide bond formation, glycosylation, lipidation, acetylation, phosphorylation, or any other manipulation or modification, such as conjugation with a labeling or bioactive component.

As used herein, the term “recombinant” in the context of a polynucleotide means a polynucleotide with semisynthetic or synthetic origin which either does not occur in nature or is linked to another polynucleotide in an arrangement not found in nature.

“Heterologous” means derived from a genetically distinct entity from the rest of the entity to which it is being compared. For example, a polynucleotide may be placed by genetic engineering techniques into a plasmid or vector derived from a different source, and is a heterologous polynucleotide. A promoter removed from its native coding sequence and operatively linked to a coding sequence other than the native sequence is a heterologous promoter.

As used herein, a “vaccine” is an immunogenic composition that is suitable for administration to an animal which, upon administration to the animal, induces an immune response strong enough to minimally aid in the protection from a clinical disease arising from an infection with a wild-type pathogenic micro-organism (e.g., strong enough for aiding in the curing of, ameliorating of, protection against, and/or prevention of a clinical disease and/or clinical signs associated therewith).

Compositions of Matter.

The present disclosure provides aroA mutant Bordetella bronchiseptica bacteria. The aroA mutant Bordetella bronchiseptica bacteria have a genetic alteration in their aroA gene, the genetic alteration being relative to the parent strain aroA gene (e.g. a virulent wild-type parent strain's aroA gene which exhibits normal aroA gene function). The genetic alteration can be effective to reduce or abolish the expression and/or biological activity of polypeptide(s) or protein(s) encoded by the aroA gene, preferably polypeptide(s) and protein(s) associated with virulence such as 3-phosphoshikimate 1-carboxyvinyltransferase. The genetic alteration can be effective to attenuate the virulence of the bacterium (e.g., reduce or abolish the pathogenicity of the bacteria). In embodiments, the genetic alteration does not materially impact the bacteria's ability to stimulate a strong and long-lasting immune response when administered to a host, even though the bacterium is attenuated (e.g., an immune response effective to provide protection against subsequent challenge with B. bronchiseptica).

The genetic alterations of the present disclosure may be made within a coding sequence to disrupt aroA gene function; however, the genetic alterations need not be located within a coding sequence to disrupt aroA gene function. The genetic alterations can also be made in nucleotide sequences involved in the regulation of aroA gene expression, for instance, in regions that regulate transcription initiation, translation, and transcription termination. Thus also included are promoters and ribosome binding regions (in general these regulatory elements lie approximately between 60 and 250 nucleotides upstream of the start codon of the coding sequence; Doree S M et al., J. Bacteriol. 2001, 183(6): 1983-9; Pandher K et al., Infect. Imm. 1998, 66(12): 5613-9; Chung J Y et al., FEMS Microbiol letters 1998, 166: 289-296), transcription terminators (in general the terminator is located within approximately 50 nucleotides downstream of the stop codon of the coding sequence or gene; Ward C K et al., Infect. Imm. 1998, 66(7): 3326-36). In the case of an operon, such regulatory regions may be located in a greater distance upstream of the coding sequence.

In embodiments, the genetic alteration includes the deletion of one or more nucleotides from a parent strain aroA gene, the substitution of one or more different nucleotides than those existing in the parent strain aroA gene, and/or the insertion of one or more nucleotides into the parent strain aroA gene. In embodiments, the genetic alteration is a partial deletion of the parent strain aroA gene (e.g. the mutant genome has a partial aroA gene). In embodiments, the genetic alteration is a complete deletion of the parent strain aroA gene (e.g., the mutant genome does not have an aroA gene).

Preferably, the parent strain has an aroA gene exhibiting normal structure and function (e.g., a structure and function consistent with wild-type virulent B. bronchiseptica strains). Suitable parent strains for use in the present disclosure include, for example, B. bronchiseptica strains exhibiting most, and preferably all, of the characteristics of strain 05 described in the examples below.

In embodiments, the aroA mutant Bordetella bronchiseptica bacteria may exhibit reduced expression of aroA gene encoded polypeptide(s) relative to a parent strain, or they may not express aroA gene encoded polypeptide(s) at all. In embodiments, the aroA mutant Bordetella bronchiseptica bacteria has less than 5% residual aroA expression after the genetic alteration relative to the parent strain, meaning that the mutant bacteria expresses less than 5% of aroA gene encoded polypeptide(s) relative to a parent strain. In embodiments, the aroA mutant Bordetella bronchiseptica bacteria expresses level(s) of aroA polypeptide(s) that are undetectable.

In embodiments, the aroA mutant Bordetella bronchiseptica bacteria express mutant aroA gene encoded polypeptide(s) having reduced biological activity relative to the parent strain's aroA gene encoded polypeptide(s), or they express mutant aroA gene encoded polypeptide(s) may have no biological activity (e.g., completely non-functional).

In embodiments, the aroA mutant Bordetella bronchiseptica bacteria express mutant aroA gene encoded polypeptide(s) having less than 5% residual biological activity after the genetic alteration relative to the parent strain's aroA gene encoded polypeptide(s), meaning that the mutant aroA gene encoded polypeptide(s) have less than 5% of the biological activity of reference aroA gene encoded polypeptide(s) from the parent strain. In embodiments, the aroA mutant Bordetella bronchiseptica bacteria express aroA gene encoded polypeptide(s) that have undetectable levels of biological activity.

In embodiments, the aroA mutant Bordetella bronchiseptica bacteria comprise a polynucleotide having at least 75%, 80%, 85%, 90%, 95%, 96%, 97%, 98%, 99%, 99.1%, 99.2%, 99.3%, 99.4%, 99.5%, 99.6%, 99.7%, 99.8%, or 99.9% identity to the sequence as set forth in SEQ ID NO:3, or a polynucleotide having 100% identity to the sequence as set forth in SEQ ID NO:3. In embodiments, the aroA mutant Bordetella bronchiseptica bacteria have an aroA locus comprising a polynucleotide having at least 75%, 80%, 85%, 90%, 95%, 96%, 97%, 98%, 99%, 99.1%, 99.2%, 99.3%, 99.4%, 99.5%, 99.6%, 99.7%, 99.8%, or 99.9% identity to the sequence as set forth in SEQ ID NO:3, or a polynucleotide having 100% identity to the sequence as set forth in SEQ ID NO:3. In embodiments, the aroA mutant Bordetella bronchiseptica bacteria comprising a polynucleotide with a certain sequence identity to SEQ ID NO:3 preferably have an attenuated phenotype, are capable of safely eliciting a protective immune response in an animal when orally administered, and/or encode for the same functionality as that encoded by SEQ ID NO: 3. Examples of comparable functions include the ability/inability to catalyze the same enzymatic reactions and the ability/inability to serve the same structural role.

In embodiments, the aroA mutant Bordetella bronchiseptica bacteria exhibit hemolytic activity. In embodiments, the aroA mutant Bordetella bronchiseptica bacteria are attenuated.

In embodiments, the aroA mutant Bordetella bronchiseptica bacteria is the strain deposited with the Collection Nationale de Cultures de Microorganismes (CNCM) under deposit number 1-5391 on Dec. 20, 2018.

In embodiments, the aroA mutant Bordetella bronchiseptica bacteria are cultured in a non-animal based culture medium and therefore free from substances of animal origin. In embodiments, the non-animal based culture medium is Tryptic Soy Broth non-animal (TSB-NA) medium. In embodiments, culturing the aroA mutant Bordetella bronchiseptica bacteria in non-animal based culture medium, such as TSB-NA, decreases the risk of contamination by adventitious agents relative to culturing the mutant bacteria in medium containing substances of animal origin. In embodiments, culturing the aroA mutant Bordetella bronchiseptica bacteria in non-animal based culture medium does not negatively impact the mutant bacteria's ability to elicit a protective immune response in an animal when orally administered.

The present disclosure also provides immunogenic compositions and vaccines comprising any aroA mutant Bordetella bronchiseptica bacteria according to the present disclosure. In embodiments, the immunogenic compositions and vaccines are effective to elicit, induce, and/or stimulate an immune response in an animal, such as a canine or feline, when administered to the animal. In embodiments, the immunogenic compositions and vaccines comprise an attenuated, aroA mutant Bordetella bronchiseptica bacteria.

In embodiments, the immunogenic compositions and vaccines are monovalent, having an aroA mutant Bordetella bronchiseptica bacteria as the lone antigen.

In embodiments, the immunogenic compositions and vaccines are multivalent, having two or more antigens, provided that at least one of the antigens is an aroA mutant Bordetella bronchiseptica bacteria according to the present disclosure. In embodiments, the multivalent immunogenic compositions and vaccines comprise, as a second antigen, a non-Bordetella bronchiseptica antigen. In embodiments, the multivalent immunogenic compositions and vaccines comprise, as a second antigen, a non-Bordetella bronchiseptica canine antigen.

In embodiments, the multivalent immunogenic compositions and vaccines comprise, as a second antigen, a canine parainfluenza virus (CPIV or PIV5) antigen. In embodiments, the canine parainfluenza virus antigen is an inactivated and/or attenuated whole virus. Examples of suitable suitable CPIV viruses for use as antigens in the present disclosure include those listed in in Table 1 below, which is derived from Rima et al.. which provides multiple sequences of canine PIV5 (Rima, B. K., et al. “Stability of the Parainfluenza Virus 5 Genome Revealed by Deep Sequencing of Strains Isolated from Different Hosts and Following Passage in Cell Culture.” Journal of Virology, vol. 88, no. 7, 2014, pp. 3826-3836, doi: 10.1128/jvi.0.03351-13). Each of the GenBank Accession Nos. of Table 1 as well as Rima et al., are incorporated by reference herein in their entirety. CPIV sequences are also disclosed in WO 2000/77043 A2, Fischer, et al., incorporated by reference herein in its entirety. As described in Rima et al., Journal of Virology, vol. 88, no. 7, 2014, canine parainfluenza viruses are highly conserved, therefore, any of the below may be used in the present invention.

TABLE 1
CPIV sequences (CPIV is sometimes referred to as PIV5,
but it is the same virus).

| PIV5 | Host | Country of | Decade of | GenBank |
|---|---|---|---|---|
| Strain | source | Origin | Isolation | accession No. |
| CPI⁺ | Dog | Germany | 1980s | JQ743321.1 |
| CPI⁻ | Dog | Germany | 1980s | JQ743320.1 |
| 1168-1 | Dog | South Korea | 2000s | KC237064.1 |
| 78524 | Dog | UK | 1980s | JQ743319.1 |
| H221 | Dog | UK | 1980s | JQ743323.1 |
| 08-1990 | Dog | South Korea | 2000s | KC237063.1 |
| D277 | Dog | South Korea | 2000s | KC237065.1 |

In embodiments, the multivalent immunogenic compositions and vaccines comprise, as a second antigen, a canine adenovirus (CAV) antigen. In embodiments, the canine adenovirus antigen is an inactivated and/or attenuated whole virus. Examples of suitable CAV viruses for use as antigens in the present disclosure include canine adenovirus type 1 (CAV-1) and canine adenovirus type 2 (CAV-2). Antigens from these pathogens for use in the vaccine compositions of the present invention can be in the form of a modified live viral preparation or an inactivated viral preparation. Methods of attenuating virulent strains of these viruses and methods of making an inactivated viral preparation are known in the art and are described in, e.g., U.S. Pat. Nos. 4,567,042 and 4,567,043. Alternatively, immunogens or antigens of CAV2, or epitopes of CAV2 immunogens, such as capsid, matrix, or hexon proteins, can be used.

In embodiments, the vaccines of the present disclosure are formulated such that they safe and effective to elicit protective immunity against B. bronchiseptica when administered to an animal, and thereby reduce and/or prevent clinical symptoms associated with subsequent B. bronchiseptica infection and disease. In embodiments, the vaccines of the present disclosure are capable of eliciting protective immune responses that are effective to decrease the gravity of B. bronchiseptica clinical signs and lesions, decrease the growth rate of B. bronchiseptica, and/or prevent death when later exposed to B. bronchiseptica.

In embodiments, the immunogenic compositions and vaccines comprise a pharmaceutically or veterinarily acceptable carrier, adjuvant, vehicle, and/or excipient. The pharmaceutically or veterinarily acceptable carriers, adjuvants, vehicles, or excipients may be any compound or combination of compounds facilitating the effective administration of the aroA mutant Bordetella bronchiseptica bacteria.

Pharmaceutically or veterinarily acceptable carriers, adjuvants, vehicles, and/or excipients are well known to one skilled in the art. For example, a pharmaceutically or veterinarily acceptable carrier, vehicle, or excipient can be a 0.9% NaCl (e.g., saline) solution or a phosphate buffer. Other pharmaceutically or veterinarily acceptable carriers, adjuvants, vehicles, or excipients that can be used include, but are not limited to, poly-(L-glutamate) or polyvinylpyrrolidone. Suitable adjuvants can include: (1) polymers of acrylic or methacrylic acid, maleic anhydride, and alkenyl derivative polymers, (2) immunostimulating sequences (ISS), such as oligodeoxyribonucleotide sequences having one or more non-methylated CpG units (“CpG Motifs Present in Bacterial DNA Rapidly Induce Lymphocytes to Secrete Interleukin 6, Interleukin 12 and Interferon γ.” Molecular Medicine Today, vol. 2, no. 6, 1996, p. 233; WO98/16247), (3) an oil in water emulsion, such as the SPT emulsion described on page 147 of “Vaccine Design, The Subunit and Adjuvant Approach” published by M. Powell, M. Newman, Plenum Press 1995, and the emulsion MF59 described on page 183 of the same work, (4) cationic lipids containing a quaternary ammonium salt, e.g., DDA (5) cytokines, (6) aluminum hydroxide or aluminum phosphate, (7) saponin, or (8) any combinations or mixtures thereof.

In embodiments, the immunogenic compositions and vaccines comprise a mucosal adjuvant which promotes improved absorption through mucosal linings. Some examples of mucosal adjuvants include chitosan, MPL, LTK63, toxins, PLG microparticles, and several others (Vajdy, M. Immunology and Cell Biology (2004) 82, 617-627; Lubben, Inez M Van Der, et al. “Chitosan and Its Derivatives in Mucosal Drug and Vaccine Delivery.” European Journal of Pharmaceutical Sciences, vol. 14, no. 3, 2001, pp. 201-207; Patel et al. “Chitosan: A Unique Pharmaceutical Excipient.” Drug Delivery Technol., 2005; Majithiya et al. “Enhancement of Mucoadhesion by Blending Anionic, Cationic and Nonionic Polymers.” Drug Delivery Technol., 2008 or Majithiya, J., et al. “Efficacy of Isavuconazole, Voriconazole and Fluconazole in Temporarily Neutropenic Murine Models of Disseminated Candida Tropicalis and Candida Krusei.” Journal of Antimicrobial Chemotherapy, vol. 63, no. 1, 2008, pp. 161-166; U.S. Pat. No. 5,980,912).

In embodiments, the immunogenic compositions and vaccines are adjuvant free (i.e., contain no adjuvant), and are effective and safe when administered to animals. In embodiments, the immunogenic compositions and vaccines are free from substances of animal origin (i.e., contain no substances of animal origin).

In embodiments, the immunogenic compositions and vaccines are formulated for one shot administration (e.g. a single administration of one dosage form). In embodiments, the immunogenic compositions and vaccines are formulated for multi-shot administration (e.g. multiple administrations of a single dosage form, multiple administrations of multiplate dosage forms).

In embodiments, the immunogenic compositions and vaccines are formulated for oral administration to an animal, such as a canine or a feline. In embodiments, the immunogenic compositions and vaccines are formulated as liquid doses for oral administration. In embodiments, the liquid dosage forms may be a liquid in a bottle or a pippete. In embodiments, the liquid dosage forms may have a dose volume generally between 0.1 to 10.0 mL, between 0.2 to 5.0 mL, between 0.1 to 1.0 mL, or between 0.5 mL to 1.0 mL. The volume of one dose refers to the total volume of immunogenic composition or vaccine administered at once to one animal.

In embodiments, the liquid dosage forms may comprise aroA mutant bronchiseptica bacteria in an amount between 1×10³ CFU to 1×10¹⁰ CFU of per dose, between 1×10⁴ CFU to 1×10⁶ CFU per dose, between 1×10⁶ CFU to 1×10⁸ CFU per dose, between 1×10⁸ CFU to 1×10¹⁰ CFU per dose, between 1×10⁴ CFU to 1×10⁵ CFU per dose, between 1×10⁵ CFU to 1×10⁶ CFU per dose, between 1×10⁶ CFU to 1×10⁷ CFU per dose, between 1×10⁷ CFU to 1×10⁸ CFU per dose, between 1×10⁸ CFU to 1×10⁹ CFU per dose, or between 1×10⁹ CFU to 1×10¹⁰ CFU per dose.

In embodiments, the liquid dosage forms may comprise a canine parainfluenza virus antigen, such as a live or attenuated whole canine parainfluenza virus, in an amount between about 6 log 10 DICC50 to about 8 log 10 DICC50 per dose, and preferably in the range of 6.7 log 10 to about 7 log 10 DICC50 per dose.

In embodiments, the liquid dosage forms may comprise a canine adenovirus antigen, such as a live or attenuated whole canine adenovirus, in an amount between. The attenuated CAV-2 should be in an amount of at least about 6 log 10 DICC50 to about 8 log 10 DICC50 per dose, and preferably in the range of 6.5 log 10 to about 6.7 log 10 DICC50 per dose.

The present disclosure also provides kits comprising aroA mutant Bordetella bronchiseptica bacteria. In embodiments, the kit comprises a vial containing any aroA mutant Bordetella bronchiseptica bacteria, immunogenic composition, or vaccine as described herein. In embodiments, the kit is intended for use with a prime-boost administration regimen and comprises a first vial containing a first vaccine or immunogenic composition of the disclosure for use in a prime administration step and a second vial containing a second vaccine or immunogenic composition of the disclosure for use in a boost administration step (the first and second vaccine or immunogenic composition may be the same, or they may be different).

Methods of Use

The present disclosure also provides methods for eliciting an immune response in an animal using the aroA mutant Bordetella bronchiseptica bacteria, immunogenic compositions, and/or vaccines of the present disclosure. In embodiments, the animal is an adult. In embodiments, the animal is a juvenile. In embodiments, the animal is between 0 and 6 months in age, between 1 and 4 months in age, or between 2 and 4 months in age.

In embodiments, provided is a method for eliciting an immune response against B. bronchiseptica in an animal comprising administering to the animal an immunogenic composition comprising an aroA mutant Bordetella bronchiseptica bacteria. In embodiments, the immunogenic composition further comprises a pharmaceutically or veterinarily acceptable carrier, adjuvant, vehicle, and/or excipient. In embodiments, the animal is canine or feline. In embodiments, the immunogenic composition is administered orally. In embodiments, the immunogenic composition is adjuvant free. In embodiments, the aroA mutant Bordetella bronchiseptica bacteria is attenuated.

In embodiments, provided is a method for eliciting a protective immune response against B. bronchiseptica in an animal comprising administering to the animal an vaccine comprising an effective amount of an aroA mutant Bordetella bronchiseptica bacteria. In embodiments, the vaccine further comprises a pharmaceutically or veterinarily acceptable carrier, adjuvant, vehicle, and/or excipient. In embodiments, the animal is canine or feline. In embodiments, the vaccine is administered orally. In embodiments, the vaccine is adjuvant free. In embodiments, the aroA mutant Bordetella bronchiseptica bacteria is attenuated. In embodiments, the animal is vaccinated/immunized against Bordetella bronchiseptica. In embodiments, the protective immune response is effective to provide the animal with protection against subsequent virulent B. bronchiseptica infection, and clinical disease and symptoms associated therewith.

In embodiments, the methods for eliciting an immune response and the methods for eliciting a protective immune response can employ a prime-boost regimen. A prime-boost regimen comprises a primary administration and a booster administration. Typically the immunological composition or vaccine used in the primary administration is different in nature from that used in the booster administration. However, the same composition/vaccine can be used in primary administration and the booster administration. In embodiments, a prime-boost regimen utilizes administrations that are preferably carried out 1 to 6 weeks apart, 2 to 5 weeks apart, 2 to 3 weeks apart, or 3 to 4 weeks apart.

Methods of Manufacture

The present disclosure also provides methods of making an aroA mutant Bordetella bronchiseptica bacteria. In embodiments, the method of making an aroA mutant Bordetella bronchiseptica bacteria comprises one or more of the following steps: (i) introducing a genetic alteration in the aroA gene of a Bordetella bronchiseptica bacteria parent strain that results in impairment of the chorismate biosynthetic pathway of the bacterium; and/or (ii) isolating an aroA mutant Bordetella bronchiseptica bacteria from the Bordetella bronchiseptica bacteria parent strain. In embodiments, the genetic alteration includes the deletion of one or more nucleotides from a parent strain aroA gene, the substitution of one or more different nucleotides than those existing in the parent strain aroA gene, and/or the insertion of one or more nucleotides into the a parent strain aroA gene. In embodiments, the genetic alteration is a partial deletion of the parent strain aroA gene (e.g. the mutant genome has a partial aroA gene). In embodiments, the genetic alteration is a complete deletion of the parent strain aroA gene (e.g., the mutant genome does not have an aroA gene). Preferably, the parent strain has an aroA gene exhibiting normal structure and function (e.g., a structure and function consistent with wild-type virulent B. bronchiseptica strains). Suitable parent strains for use in the present disclosure include, for example, B. bronchiseptica strains exhibiting most, and preferably all, of the characteristics of strain 05 described in the examples below.

The present disclosure also provides methods of propagating an aroA mutant Bordetella bronchiseptica bacteria. In embodiments, the methods include culturing an aroA mutant Bordetella bronchiseptica bacteria in a non-animal based medium. In embodiments, the non-animal based culture media is Tryptic Soy Broth non animal (TSB-NA).

The invention will now be further described by way of the following non-limiting examples.

### EXAMPLES

### Example 1—Characterization of B. bronchiseptica Strains

Various isolated strains were reviewed to determine which ones would be useful in the development of a new canine injectable (inactivated, attenuated, or both) Bordetella bronchiseptica vaccine. Characterization of sixteen (16) isolates from an internal collection was conducted, including an evaluation of growth, hemolytic activity, regulation of virulence/immunogenic factors production and molecular typing. The potential for each of the strains to be a safe and effective component of a vaccine formulation was evaluated.

Sixteen Merial B. bronchiseptica isolates of pig, dog and human origin (listed in Table 2), were selected for the characterization. For comparison, two control strains were included in all experiments.

TABLE 2
Sixteen B bronchiseptica isolates and their origins

| Strain reference No. | Origin |
|---|---|
| 01 | Human |
| 02 | Dog |
| 03 | Dog |
| 04 | Pig |
| 05 | Pig |
| 06 | Pig |
| 07 | Pig |
| 08 | Dog |
| 09 | Dog |
| 10 | US strain (372CN) |
| 11 | Dog |
| 12 | Dog |
| 13 | Pig |
| 14 | Unknown |
| 15 | Unknown |
| 16 | Dog |
| Control 1 | Dog (286, CNR collection) |
| Control 2 | Rabbit (RB50, US, sequenced genome) |

TABLE 3
Description of the tests

| Test | Principle | Strains tested |
|---|---|---|
| Hemolysis | detection of a halo upon growth on solid rich BG | All |
| Adenylate cyclase | determination of ATP transformation into AMPc | All |
| activity | due to the action of the calmodulin dependent | |
| | AC-Hly upon growth on solid rich BG | |
| Detection of the | Western blotting using mice polyclonal | All |
| production of | sera raised against purified B. bronchiseptica | |
| antigenic/virulence | BteA and purified B. pertussis FHA, PRN and | |
| factors | AC-Hly | |
| | After growth of the isolates on | |
| | i) solid rich Bordet Gengou medium at 37° C. or | All |
| | 25° C. | |
| | ii) enriched liquid Stainer Scholte (SS) medium | 01, 02, 03, 04, 05, |
| | at 37° C. with or without the addition of MgSO4 | 06, 07, 08, 09, and |
| | | 10 |
| | iii) Tryptic Soy Broth (TSB) liquid medium at | 06, 09, and 10 |
| | 37° C. | |
| Fimbria typing | Agglutination of a bacterial suspension upon | All |
| | incubation with mice polyclonal anti Fim2 or | |
| | Fim3 antibodies raised against purified fimbriae | |
| Determination of | Culturing in enriched SS medium | 1, 02, 03, 04, 05, |
| the generation time | | 06, 07, 08, 09, and |
| | | 10 |
| Mobility assays | Picking isolated bacterial colonies on semi- | All |
| | synthetic, semi-solid Cyclodextrin medium | |
| | (CSM), at 25° C. or 36° C. with or without the | |
| | addition of MgSO4 or Nicotinic acid | |
| Typing | Pulsed Field Gel Electrophoresis (PFGE) and | All |
| | Multi Locus Sequencing Typing (MLST). | |
| cya locus | PCR at the cya locus encoding adenylate cyclase | 2, 03, 11, and 12 |
| replacement | | |
| LD50 on mice | Challenge of groups of 10 mice with 3 × 10⁸, | 04, 05, 06, 07, 09, |
| | 3 × 10⁷ and 3.10⁶ cfu/ml of B. bronchiseptica | and 10 |
| | and observation of mortality during 15 days | |

Ten out of sixteen strains showed hemolysis at 37° C. Six out of sixteen isolates were non-hemolytic (02, 03, 11, 12, 15 and 16). These data correlate with the measurement of adenylate cyclase activity and immunodetection of AC-HLY protein. Strains 02, 03, 11, 12 lack a cya gene, while 15 and 16 are may be locked in a non-virulent phase IV.

The results showed that all tested isolates grew on enriched media and there was no significant difference of generation time amongst them in this medium. BteA and FHA were produced by all isolates, except 15 and 16, in classical vag expressing conditions. PRN was produced by all isolates except 06, 10, 15 and 16. Production of virulence factors in rich BG or synthetic SS medium was similar for all isolates for which both media were tested. The regulation of virulence factors expression is “classical” for twelve out of the sixteen strains, with repression at 25° C. and in the presence of MgSO4 or nicotinic acid. Two strains 09 and 05 showed partial constitutive expression of virulence factors respectively. Two strains 15 and 16 showed complete repression of virulence factors production. They are considered to have switched to an avirulent phase IV (Monack et al., 1989, Mol. Microbiol. 3: 1719), meaning that they are locked in a vag repressing phase, not expressing any virulence determinants whatever the conditions tested. The growth on TSB was 2 to 3× faster than in SS medium for tested isolates in these media. The production of BteA and PRN seems overall lower in TSB compared to SS. Fim 2 is produced by eight isolates from the sixteen isolates. None of the isolates produced Fim 3.

The results showed that ten isolates exhibited a “classical” regulation of motility, being motile at 25° C. but not at 37° C. Strain 05 showed a mixed motility phenotype: motility is de-repressed in certain conditions only (MgSO4 or 25° C. respectively) which correlates with the semi-constitutive expression of vag genes (no systematic de-repression of vrg genes). Both 06 and 10 were non-motile likely due to a mutation in fla genes. 15 and 16 are constitutively motile, in coherence with the above observation that they are locked in a vrg expression, avirulent phase IV.

The sixteen isolates clustered into 7 different PGFE groups (A to G) and four different sequence types or ST. Strains 15 and 16 grouped together with reference RB50 of rabbit origin. Strains 02, 03, 11, 12 belong to ST27. One particularity of this lineage is the replacement of the cya locus by ptp as described (Buboltz et al. 2008. J. Bacteriol. 190:5502). Accordingly, these 4 strains did not display hemolytic activity but rather, an otherwise “classical behavior” in terms of production of other virulence factors. The cya locus replacement by the ptp operon was confirmed by PCR for these strains.

The molecular typing study and sequence analysis are further described in Example 2 below.

The results of LD50 on Mice are shown in Table 4 below.

TABLE 4
LD50 on Mice

| | CFU/ml | CFU/mouse |
|---|---|---|
| Control 1 | 7 × 10⁵ | 3.5 × 10⁴ |
| 04 | 1.7 × 10⁷ | 8 × 10⁵ |
| 05 | 9 × 10⁶ | 4.5 × 10⁵ |
| 09 | 4.5 × 10⁶ | 2.25 × 10⁵ |

All strains tested showed similar virulence profiles, independent of their constitutive regulation, or absence of flagella and pertactin.

Conclusion. Strains 05 and 09 were deemed to be strong vaccine strain candidates because they are both partially or fully constitutive for the expression of virulence factors that are also important antigenic determinants. Strains 02, 03, 11, and 12 lack the cya locus, a typical feature of ST27 strains and therefore do not display any hemolytic activity. Further, both 06 and 10 are non-motile and do not express pertactin, but nonetheless remain virulent. Importantly, all analyses show that the 06 and 10 vaccinal strains behave identically, indicating no major change in the strain background upon passages.

Strains 15 and 16 were constitutively motile and did not express any virulence factor whatever the tested conditions. They were considered to have switched to an avirulent phase IV, meaning that they were locked in a vag repressing phase, not expressing any of the other virulence factors whatever the conditions tested. These strains constitutively express virulence repressed genes, therefore showing motility in all tested conditions. As such, these strains were good candidates to be avirulent in mice.

### Example 2—Deletion of the aroA Gene in the Parental B. bronchiseptica Strain (Strain 05)

An aroA gene deletion in B. bronchiseptica creates auxotrophy in the mutant. This type of deletion mutant is not able to grow in vivo or in growth medium in vitro when not supplemented with the 3 essential aromatic amino acids for bacterial growth (Phenylalanine, tryptophan and tyrosine).

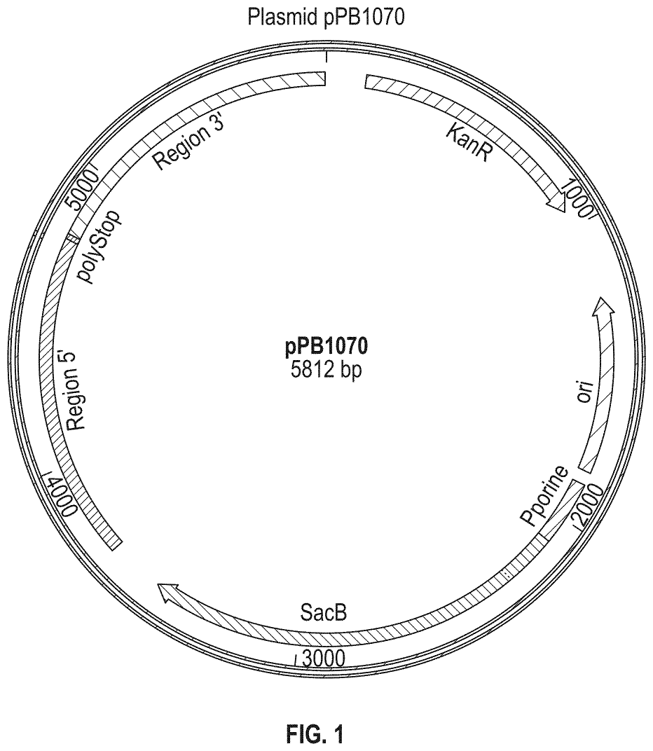

Summary. In this example, a Bordetella bronchiseptica aroA deleted mutant was generated. The methodology used to perform the genetic modification (full aroA gene deletion) was based on an engineered suicide plasmid pPB1070 (see FIG. 1). This plasmid was replicative in E. coli but not in B. bronchiseptica. This plasmid contained the ColE1 origin of replication, the kanamycin gene resistance, the sacB gene (as counter-selection system) placed under the porine promoter of B. bronchiseptica and the deletion cassette. The deletion cassette contained only the two genes flanking the aroA gene at 5′ (downstream gene) and 3′ (upstream gene). The aroA gene was replaced by a 6× polystop.

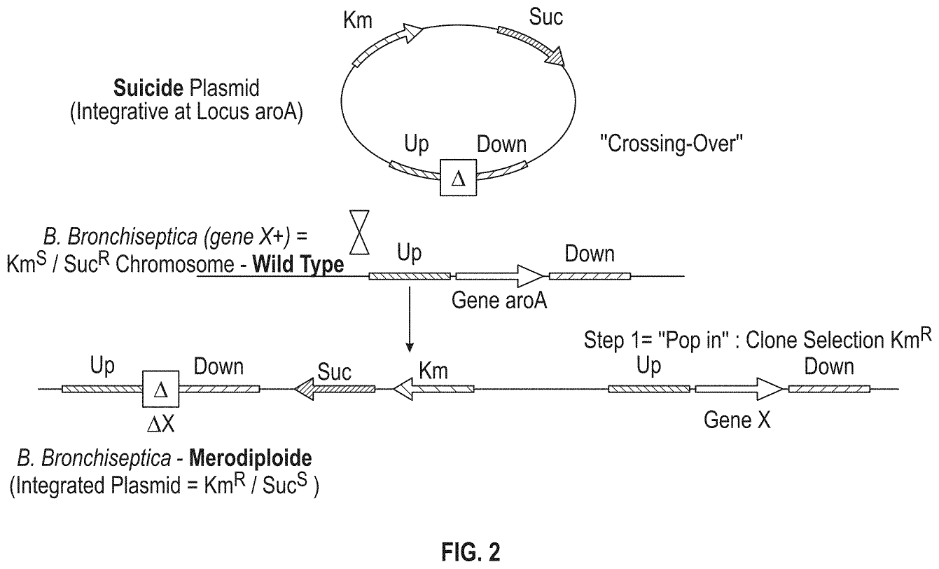

The pPB1070 plasmid was introduced into B. bronchiseptica strain 05 by electroporation and was integrated into the chromosome following a first recombination event at the aroA gene locus either at 5′ end or at 3′ end (see FIG. 2). This plasmid integration is also named “pop in”.

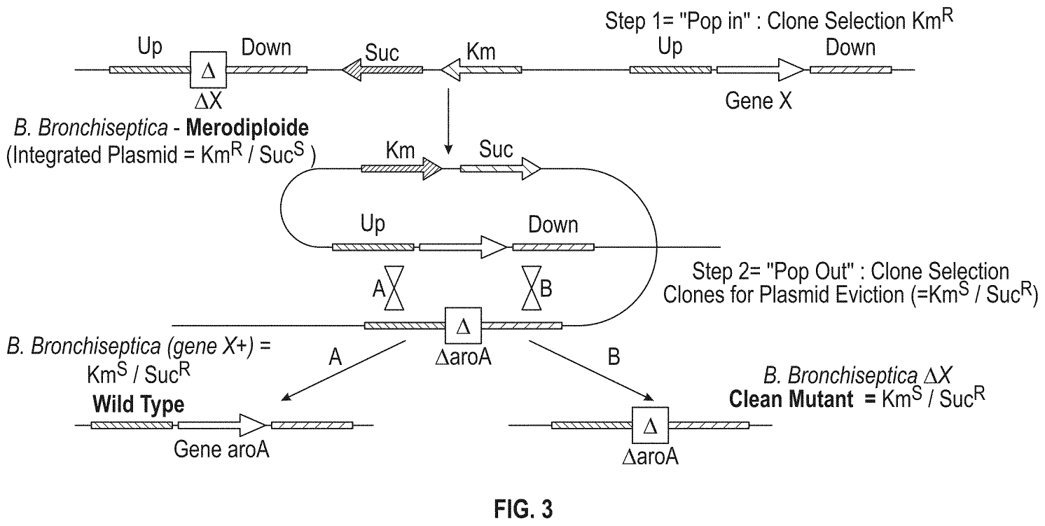

The transformant clones (integrant clones) are named Merodiploids that became resistant to the kanamycin (Km^{R}) and sensitive to sucrose (Suc^{s}). A second recombination event occurs randomly during bacterial growth leading to the possible isolation of either a wild type strain (aroA⁺) or an aroA deletion mutant (ΔaroA) which is sensitive to Kanamycin (Km^{S}) and resistant to sucrose Suc^{R}, as expected due to the loss of eviction of the pB1070 plasmid out of the chromosome following this second recombination event (see FIG. 3). Gene-specific PCR allowed the identification of the desired B. bronchiseptica aroA deleted mutant. This is confirmed by sequencing the full aroA locus: absence of the aroA gene and absence of the pPB1070 plasmid.

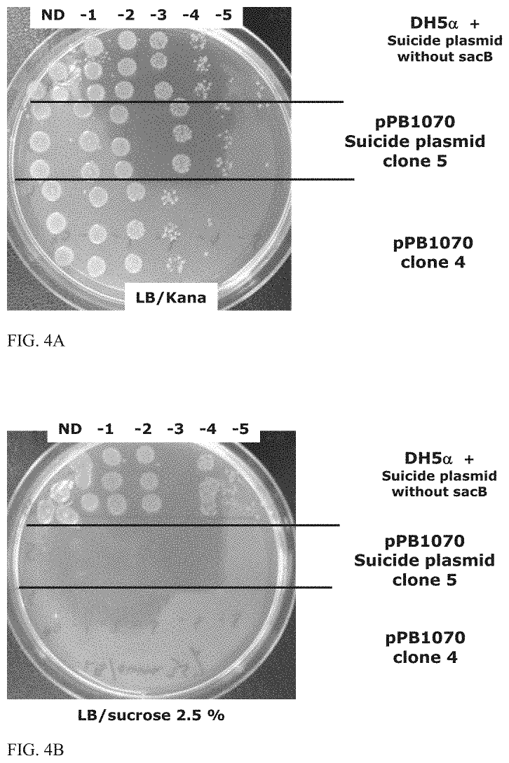

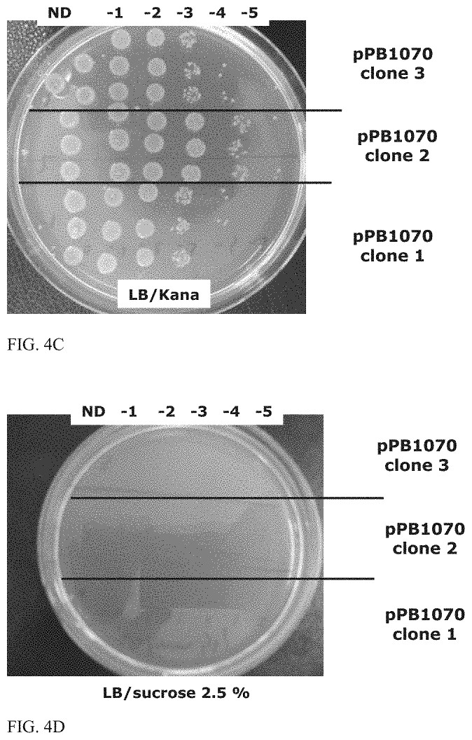

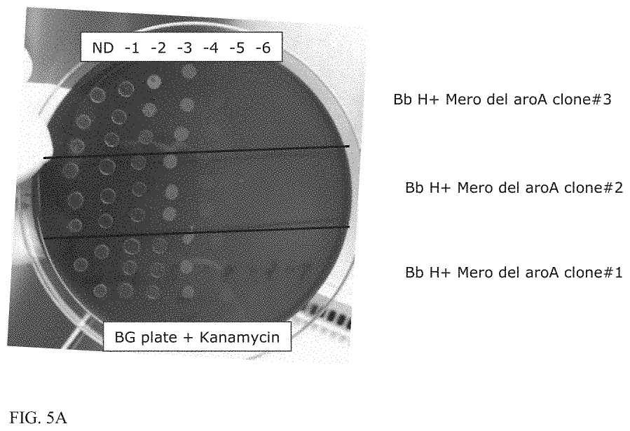

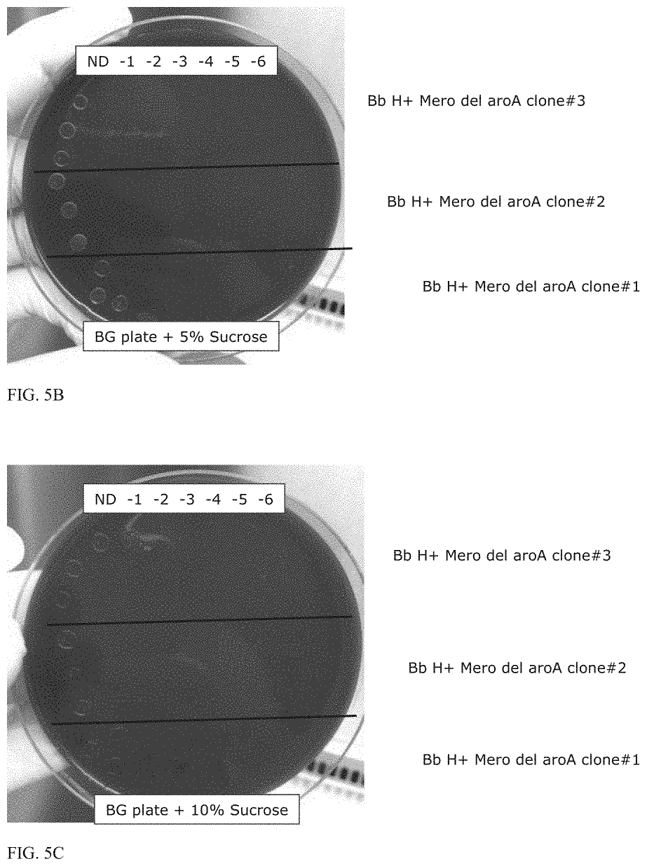

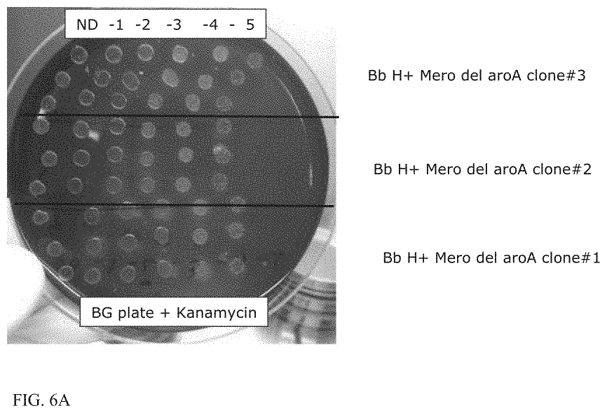

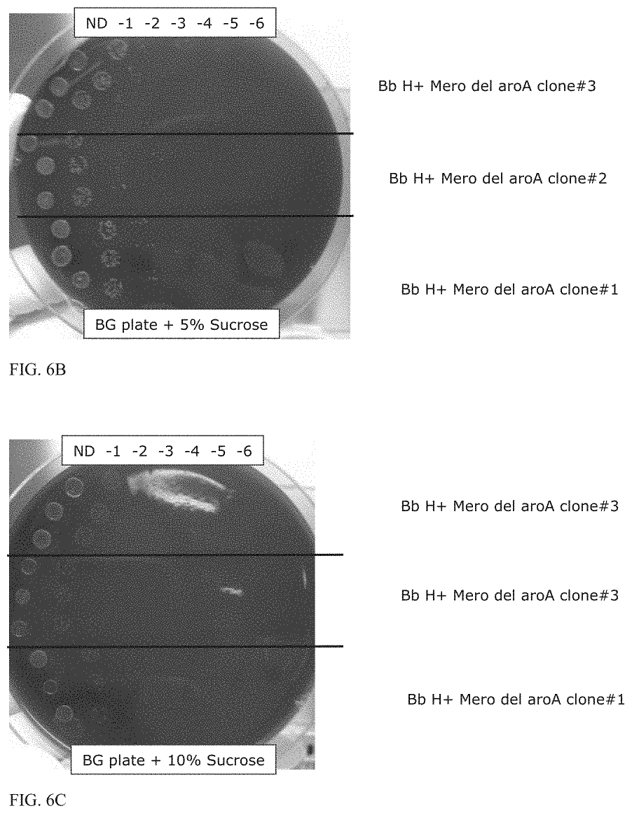

Functional characterization in E. coli of the pPB1070 suicide plasmid for aroA deletion in B. bronchiseptica. Replicative plasmid in E. coli=pluricopy effect of the SacB/Sucrose toxicity (=Counterselection system). As shown in FIGS. 4A to 4D, the suicide plasmid pPB1070 was successfully integrated into B. bronchiseptica (Bb). As such, this plasmid may be used to rapidly delete the aroA gene from other Bb strains, including the H+parental strain. Functional characterization in B. bronchiseptica of the integrated pPB1070 suicide plasmid for the aroA deletion in B. bronchiseptica (sacB placed under the control of the porine promoter). Integrated plasmid into the B. bronchiseptica chromosome=monocopy effect of the SacB/Sucrose toxicity (=Counter-selection system). As demonstrated by the results shown in FIGS. 5A-5C and FIGS. 6A-6C, a high sensitivity to sucrose equals successful functionality of the counter-selection system with greater than 5 log survival reduction of Merodiploids. Dilution & dot volume of Merodiploid clones' culture was tested and confirmed by PCR for their ability to grow on BG plate+5% or 10% sucrose.

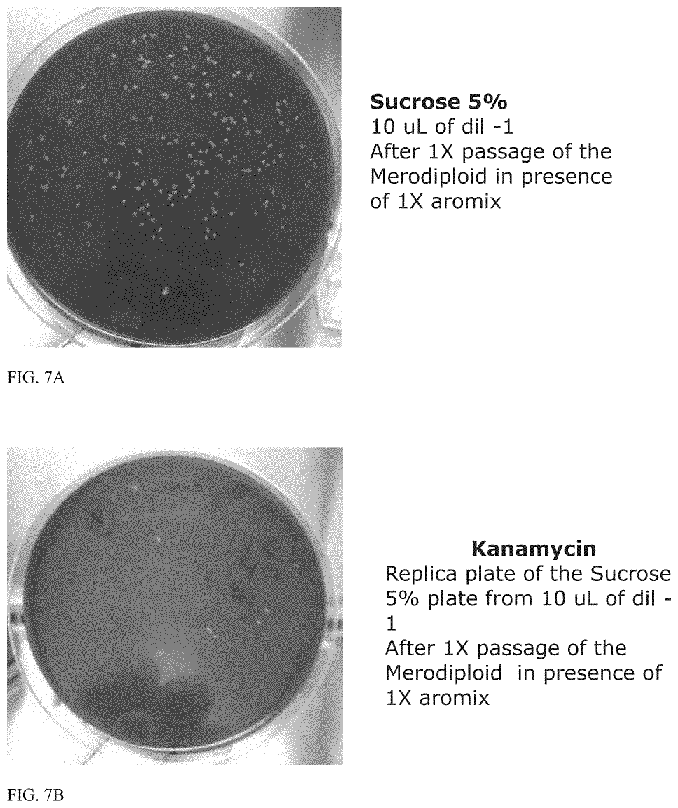

ΔaroA deletion mutant screening using the sacB/Sucrose counter-selection system. As shown in FIGS. 7A-7B, B. bronchiseptica with a high sensitivity to sucrose was demonstrated when 10 uL of a Merodiploid culture grown (1×) in BTS+aromix and spread on 5% sucrose plates gave rise to about 100 CFUs that are sucrose resistant. According to the replica plate assay on BG plate with and without Kanamycin (FIG. 7B), the non-sensitive clones represented 96% of the CFUs. Finally, SucR and KmS gene-specific PCR confirmed that the 96 CFUs were “pop out” clones giving rise to 100% hemolytic recombinants.

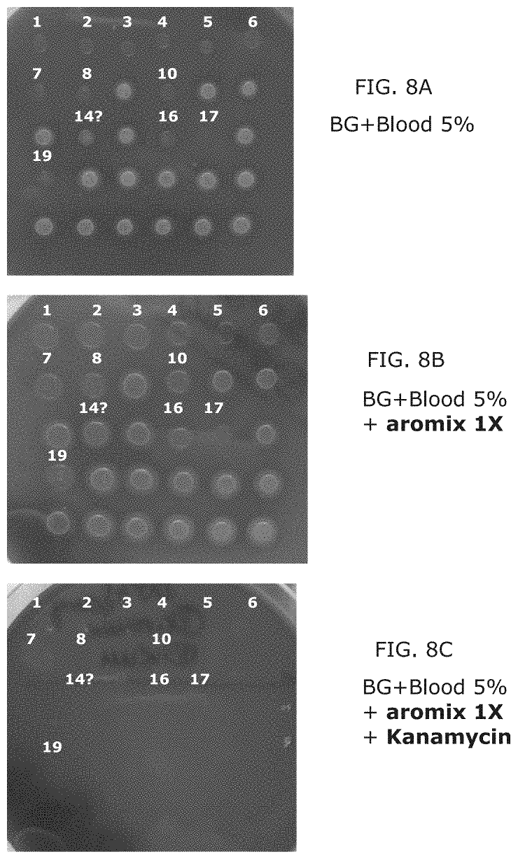

Identification of optimal aroA mutant isolation conditions. As shown in FIG. 8, Clone #2 was an aroA deleted, hemolytic H+, mutant. When aroA has been deleted from the B. bronchiseptica genome of this example, the total aroA gene locus is 2.3 kb smaller compared to the wild-type aroA gene locus, which is 3.6 kb. The frequency for obtaining one aroA gene deleted mutant H+ for B. bronchiseptica is a scant 1/10⁶ and, as such, obtaining even one aroA mutant was highly improbable absent using a technique such as the 100% functional Counter-selection system of the present example.

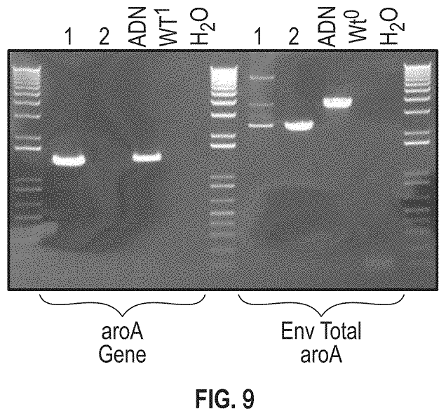

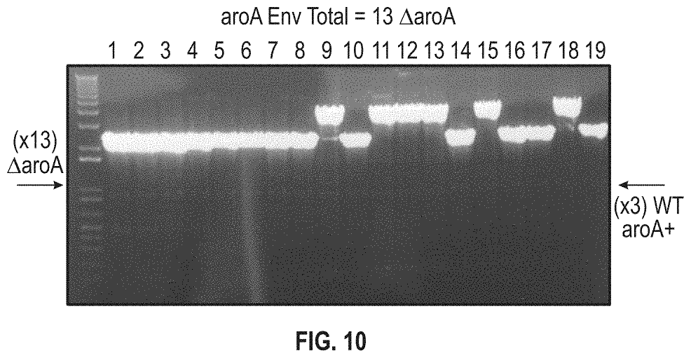

Conclusion. Thirteen (13) new aroA-gene deleted, H+, B. bronchiseptica mutants were engineered and characterized. As confirmed by PCR and/or sequencing, clones 1, 2, 3, 4, 5, 6, 7, 8, 10, 14, 16, 17 & 19 are ΔaroA deleted mutant B. bronchiseptica (see FIG. 9). All these clones are H+ and were streaked on BG+ blood supplemented with 1× and 2× of aromix for isolation of H+CFUs prior initiating a liquid culture for subsequent storage at −70° C. (see FIG. 10). In general, liquid cultures were performed from the isolated clones and culture were grown at 180 rpm at 37° C. in 1) BTS, 2) BTS+1× aromix and BTS+2× aromix, and harvested at OD₆₉₄ around 1.0-1.3. Finally, H+ activity assays were conducted from each resulting culture condition, by spreading on BG+blood+1× aromix, an aliquot 150 of 10⁻⁶ dilution.

### Example 3—Efficacy of Bb ΔaroA Oral Vaccine

Objectives & Study Overview. To evaluate the efficacy of a candidate Bordetella bronchiseptica (Bb) (L1aroA) vaccine after 2 immunizations 20 days apart in dogs negative for Bb. The low dose (5×10⁴ CFU/dose) was delivered subcutaneously, and the high dose was delivered orally (3-5×10⁹ CFU/dose). Efficacy was evaluated by a test 7 days after the last vaccination. Clinical signs were evaluated in comparison to an unvaccinated group. All dogs were determined to be negative prior to vaccination (D-2) and negative prior to challenge (T-1). Table 5 presents the experimental design.

Growth Conditions and Bacterial Titer. The aroA gene deleted, H+ mutant Bordetella bronchiseptica (L1aroA), was cultivated in liquid medium TSB (Tryptic Soy Broth) supplemented with 1× aromix at 37° C. under 200 rpm agitation until about the end of the log phase. At this point, the culture had and OD at 694 nm of about 1.2 (OD694=1.2), which corresponds to a bacterial titer close to 5×10⁹ CFU/ml. These results correspond to about 12-13 hours in culture. Next, the culture was titrated in parallel using flow cytometry, and then diluted to target titers. Vaccine bacterial counts and hemolysis were carried out on BG (Bordet Gengou) agar+5% sheep blood+1× aromix. Similarly, the purity of the vaccine was tested in parallel with the vaccination.

TABLE 5
Experimental design

| | Vaccination | Challenge | Clinical | | |
|---|---|---|---|---|---|
| Gp | D0 & D20 | D27 = T0 | monitoring | Sampling | Euthanasia T14 |
| A | Bb ΔaroA | Intranasal: | Weighing: | For qPCR: | Post-mortem |
| (n = 6) | Target titer: | 0.5 | every | D-2, D19, T-1 | samples (lungs |
| | 10⁵ UFC/ml | ml/nostril + | 2 | and T2, T3, T6, | and |
| | 0.5 ml; SQ | intra- | weeks | T8, T10, T14 | injection sites) |
| B | Bb ΔaroA | tracheal: | Clinical | Serology: | For |
| (n = 6) | target titer: | 1 ml; | monitoring: | D-2, D5, D12, D19 | histopathology |
| | 3x10⁹ | Target | Morning: D0* to D4; | T-1, T14 | |
| | UFC/ml | titer: | D20 to D24; | | |
| | 1 ml; VO | 3.10⁹ | T0* to T14 | | |
| C | NA | UFC/ml | Afternoon: | | |
| (n = 5) | | | between T0 and | | |
| | | | T13 every day | | |
| | | | except week end | | |

*before vaccination or challenge;
Bb suspensions real titers: Gp A Vacc1: 1.4 × 105 CFU/ml;
Gp A Vacc2: 0.86 × 105 CFU/ml;
Gp B Vacc1: 3.5 × 109 CFU/ml;
Gp B Vacc2: 2.61 × 109 CFU/ml.

Challenge Strain. The challenge Bb strain was amplified to prepare a G4 generation in liquid medium (TSB) at 37° C. from a 1/50th inoculation with 200 rpm shaking. The G4 amplification was stopped after about 7.30 hours, and the culture was immediately titrated via FACS and diluted to the target titer. In parallel, the culture was spread on BG agar supplemented with 5% sheep blood (Biomerieux) and incubated 48 h at 37° C. to assess the homogeneity and appearance of the colonies (e.g. smooth, small gray colonies), and also their hemolytic character. About 100% of the colonies expressed the hemolytic phenotype. In parallel, titration on agar was conducted to confirm the FACS-determined titer.

Animals. Beagle dogs, negative for Bb by qper from nasal swabs and negative for serum anti-Bb antibodies, aged between 9 and 12 weeks at Day 0. The animals were randomized and divided into 2 groups of 6 dogs and a group of 5 dogs according to their dates of birth and the qPCR Bb titers, before D0.

Prior to vaccination (D0), all Group A dogs were shaved in the interscapular zone. The vaccine strain was prepared just before the test. On the day of vaccination, 10 ml of vaccine strain at about 10⁵ CFU/ml and 3-5×10⁹ CFU/ml was prepared and set on ice. An aliquot of the inoculum was tittered (FACS and box counting). Only dogs in good general condition and without hyperthermia (except hyperthermia related to excitation: T ° C.>39.5° C. but alert and reactive) were vaccinated. Group A: At D0 and D20, all dogs in group A were vaccinated subcutaneously with 0.5 ml of the vaccine Bb aroA had about 10⁵ CFU/ml. Group B: On D0 and D20, all Group B dogs were vaccinated orally with 1 ml of Bb aroA at about 3-5×10⁹ CFU/ml. Group C animals were not vaccinated.

TABLE 6
Hyperthermia results

| Groups | Dogs | T0* | T1 | T2 | T3 | T4 | T5 | T6 | T7 | T8 | T9 | T10 | T11 | T12 | T13 | T14 |
|---|---|---|---|---|---|---|---|---|---|---|---|---|---|---|---|---|
| A | 22888 | 38.4 | 39.6 | 39.2 | 39.1 | 38.9 | 39.0 | 38.7 | 38.9 | 38.4 | 38.4 | 38.6 | 39.0 | 38.8 | 38.7 | 38.9 |
| ΔaroA | 30770 | 38.7 | 39.3 | 39.4 | 39.6 | 39.2 | 39.3 | 38.9 | 38.8 | 38.5 | 38.5 | 38.6 | 39.0 | 39.1 | 39.0 | 38.9 |
| 10\hat{ }5 | 67657 | 38.6 | 39.1 | 38.9 | 39.4 | 39.1 | 39.2 | 39.2 | 38.4 | 38.4 | 38.9 | 38.7 | 39.1 | 39.3 | 39.2 | 39.1 |
| CFU/ml | 67803 | 39.0 | 39.9 | 39.7 | 40.0 | 39.4 | 39.8 | 37.5 | 39.1 | 38.6 | 38.9 | 38.7 | 38.8 | 38.9 | 38.6 | 38.7 |
| SC | 67959 | 38.8 | 39.5 | 39.7 | 39.8 | 39.4 | 39.1 | 38.8 | 38.5 | 38.3 | 38.5 | 38.5 | 38.7 | 39.0 | 38.6 | 38.7 |
| | 72267 | 38.5 | 39.3 | 39.4 | 39.2 | 39.2 | 38.9 | 38.3 | 38.7 | 38.6 | 38.5 | 38.1 | 38.5 | 38.9 | 38.8 | 38.6 |
| B | 22710 | 38.4 | 39.3 | 39.4 | 39.8 | 38.9 | 38.9 | 38.8 | 38.8 | 38.6 | 38.8 | 38.6 | 38.8 | 38.8 | 38.9 | 38.8 |
| ΔaroA | 23213 | 38.8 | 39.1 | 39.2 | 39.2 | 38.9 | 38.9 | 38.6 | 38.7 | 38.5 | 38.7 | 38.6 | 38.3 | 38.9 | 38.7 | 38.8 |
| 3-5.10\hat{ }9 | 23353 | 39.0 | 39.1 | 39.3 | 39.3 | 38.5 | 39.0 | 39.0 | 38.7 | 39.0 | 38.9 | 38.7 | 38.8 | 39.1 | 39.1 | 39.4 |
| CFU/ml | 73097 | 38.7 | 39.1 | 39.0 | 39.4 | 39.3 | 39.8 | 39.2 | 39.2 | 38.9 | 38.5 | 38.6 | 39.2 | 39.1 | 39.6 | 39.0 |
| VO | 83332 | 38.8 | 38.9 | 38.9 | 39.0 | 38.8 | 39.1 | 39.1 | 38.7 | 38.7 | 38.8 | 38.7 | 38.7 | 39.0 | 39.1 | 39.0 |
| | 83467 | 38.7 | 39.1 | 39.4 | 39.3 | 39.3 | 39.2 | 38.9 | 38.5 | 39.1 | 38.4 | 38.6 | 38.5 | 38.7 | 39.0 | 39.0 |
| C | 22578 | 38.9 | 39.9 | 39.8 | 40.1 | 39.9 | 39.9 | 39.7 | 39.6 | 39.1 | 39.4 | 39.1 | 39.1 | 39.1 | 38.6 | 39.1 |
| Controls | 30821 | 39.3 | 40.3 | 39.7 | 39.8 | 39.4 | 39.1 | 39.0 | 39.1 | 38.6 | 39.0 | 38.8 | 39.3 | 39.1 | 38.8 | 39.4 |
| | 67404 | 38.8 | 40.4 | 40.3 | 40.2 | 39.9 | 39.5 | 39.6 | 39.3 | 38.7 | 39.1 | 38.8 | 39.3 | 39.2 | 38.9 | 39.2 |
| | 67727 | 38.8 | 40.1 | 39.8 | 39.9 | 39.7 | 40.0 | 40.1 | 40.0 | 39.6 | 39.3 | 39.1 | 39.1 | 39.4 | 38.7 | 38.9 |
| | 68132 | 39.4 | 40.2 | 39.8 | 40.1 | 40.1 | 40.0 | 39.9 | 39.4 | 39.0 | 39.5 | 38.9 | 39.4 | 39.0 | 38.6 | 39.2 |

*before challenge

All controls with hyperthermia (RT>39.5° C.) after challenge for at least 3 days. In gp A: 4 dogs/6 with hyperthermia for 1, 2 or 4 days. In gp B: 2 dogs with ponctual hyperthermia once.

TABLE 7
All Controls validated the challenge according to both the
USDA (“spontaneous cough for 2 or more cumulative days”)
and Ph. Eur. Endpoints.

| Gp | GCS |
|---|---|
| A | 156 |
| (ΔaroA 10\hat{ }5 CFU/ml SC) | |
| B | 45 |
| (ΔaroA 3.10\hat{ }9 CFU/ml VO) | |
| C (Controls) | 393 |

TABLE 8
Clinical signs after challenge (data from T0-T14). USDA
criteria: at least 75% of the control dogs develop spontaneous
cough for two or more consecutive days

| | | | | | Mean |
|---|---|---|---|---|---|
| | | No. of | % of | No. of | No. of |
| | | Dogs | Dogs | Dogs with | Days |
| | | with | with | Spontaneous | with |
| Group | Treatment | Disease | Disease | Cough | Cough |
| A | aroA 10\hat{ }5 | 4 | 80 | 4 | 2.7 |
| | CFU/ml SC | | | | |
| | (n = 6) | | | | |
| B | aroA 3 × 10\hat{ }9 | 0 | 0 | 4 | 0.7 |
| | CFU/ml VO | | | | |
| | (n = 6) | | | | |
| C | Controls | 5 | 100 | 5 | 7.4 |
| | (n = 5) | | | | |

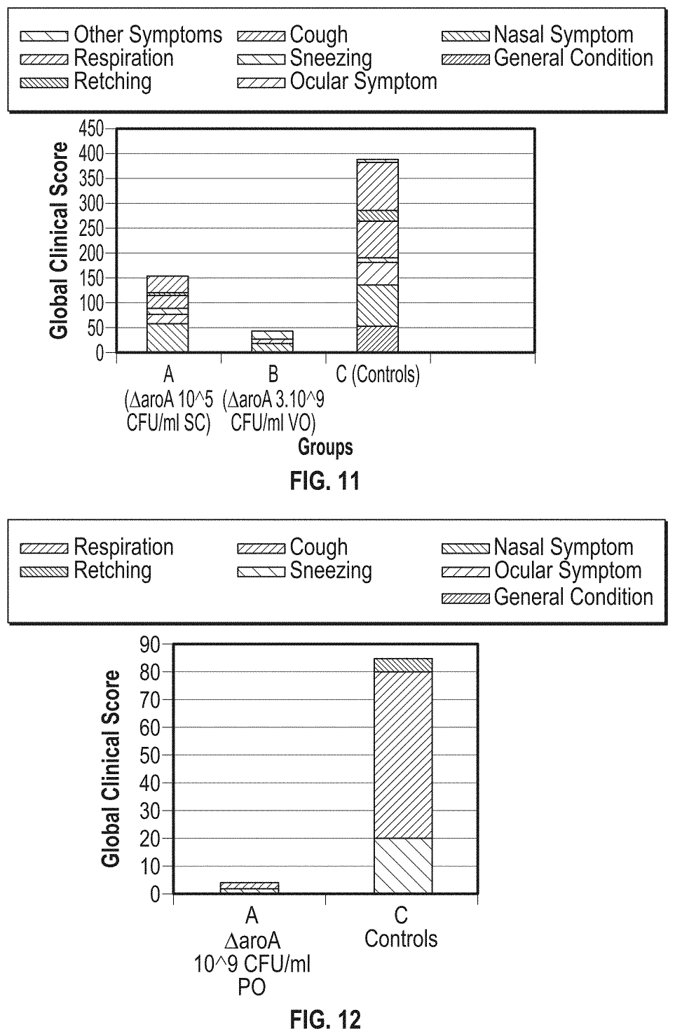

FIG. 11 shows the clinical signs in canines post administration of the indicated treatments, followed by virulent challenge with virulent B. bronchiseptica.

### Example 4—Efficacy of Oral B. bronchiseptica ΔaroA

Objectives. To assess the efficacy of one shot vaccination by oral route of a candidate vaccine (aroA-deleted Bordetella bronchiseptica (Bb ΔaroA) at high dose (10⁹ CFU/ml dose).

TABLE 9
Experimental design - single oral vaccination

| | Vaccination | Challenge | Clinical | | |
|---|---|---|---|---|---|
| Gp | D0 & D20 | D21 = T0 | monitoring | Sampling | Euthanasia T14 |
| A | Bb ΔaroA | Intranasal: | Weighing: | For qPCR: | Post-mortem |
| (n = 5) | Target titer: | 0.5 | D-1, D14, | nasal swabs: D-2, D7, | samples (lungs |
| | 10⁹ UFC/ml | ml/nostril + | D20, | D14, D20, T2, T3, | and |
| | 1.0 ml; Oral | intra- | T14 | T6, T8, T10, T14 | injection sites) |
| C | NA | tracheal: | Clinical | Serology: | For |
| (n = 5) | | 1 ml; | monitoring: | D-1, D7, D14, D20, | histopathology |
| | | Target | Morning: | T14 | |
| | | titer: | T0 to T14; | | |
| | | 1 × 10⁹ | Afternoon: | | |
| | | UFC/ml | between T0 and | | |
| | | | T13 every day | | |
| | | | except week end | | |

*before vaccination or challenge

Challenge Strain. Prepared as described above.

Animals. Beagle dogs, negative for Bb by qper from nasal swabs and negative for serum anti-Bb antibodies, aged between 9 and 12 weeks at Day 0. The animals were randomized and divided into 2 groups of 5 dogs and a group of 5 dogs according to their dates of birth and the qPCR Bb titers, before D0.

Clinical signs results. A dog is classified as having the disease due to Bb infection if it develops spontaneous cough (AM and/or PM) for two or more consecutive days (as per USDA endpoint definition).

TABLE 10
Clinical signs results

| | | | | Mean |
|---|---|---|---|---|
| | No. | % | No. Dogs | number |
| | Dogs | Dogs | with | of days |
| | with | with | spontaneous | with |
| Group | disease | disease | cough | cough |
| A | 0 | 0 | 2 | 0.4 |
| C | 4 | 80 | 5 | 6.4 |

Clinical protection against challenge was confirmed for both vaccine Groups by Global Clinical Scores (FIG. 12).

Summary. The orally-delivered vaccine containing an effective amount of the disclosed attenuated B. bronchiseptica strain was able to elicit protection.

### Example 5—Bordetella bronchiseptica: Comparison of Two Production Processes of Two ΔaroA Bivalent Vaccines

Objectives. To assess efficacy of 2 different ΔaroA vaccines (different production processes) after one shot vaccination by oral route.

The first vaccine was prepared in Cohen Wheeler (CW) medium (Cohen S M, Wheeler M W. Pertussis Vaccine Prepared with Phase-I Cultures Grown in Fluid Medium. Am J Public Health Nations Health. 1946 April; 36(4):371-376). The second vaccine was prepared in a non-animal Tryptic Soy Broth (see Example 6 below). A total of 18 SPF dogs between 9-12 weeks old were randomized into 3 groups of 6 by age and sex. Both vaccines also included live, attenuated CPIV (PIV5) antigens as described below.

TABLE 11
Experimental design—comparison of 2 ΔaroA vaccines

| | Vaccination at Day 0 | | | | | |
|---|---|---|---|---|---|---|
| | Oral route—3.3 ml | | Challenge | | | |
| | Strain | Strain | at T0 | Clinical | | Euthanasia |
| Group | Bb ΔaroA | PIC | (=D20) | endpoints | Samples | T14 |
| A | Process | Target titer: | Nebulization, | Weigh: | 2 nasal | Post mortem |
| (n = 6) | CW | 6.9log10 | target titer | D-1, D13, T-1, | swabs | samples of |
| | Target titer | DICC50/dose | ~9log10 | T14 | (qPCR): | lungs and |
| | 9log10, | | CFU/chamber | Post vaccination | D-1, D1, | trachea for |
| | CFU/dose | | Target | clinical check up: | D2, D7, T- | histological |
| B | Process | | volume to be | D0 to D4 | 1, T1, T3, | analyses |
| (n = 6) | TSB | | nebulized | Post challenge | T6, T8, | |
| | Target titer | | ~20 mL | clinical checkup: | T10, T14 | |
| | 9log10, | | | AM: D0 to D14 | 2 tracheal | |
| | CFU/dose | | | PM: between | swabs: | |
| C | NA | | | T0 and T13, | D-1, D7, T- | |
| (n = 6) | | | | every day except | 1 and T8 | |
| | | | | week-ends. | Blood (dry | |
| | | | | | vials 4 | |
| | | | | | mL): D-1, | |
| | | | | | D7, D13, | |
| | | | | | T-1 T14 | |

Final challenge dose/chamber

Preparation of Canine Parainfluenza Virus vaccine (PIC). The initial suspension (Titer 8.7 log 10 DICC50/ml) of the vial containing the PIC (CPIV) vaccine strain was homogenized by rollover of the tubes. All the cups were rebuilt in frozen ice. The titre is calculated in infectious doses for cell culture 50% (DICC50) by the Kàrber method. Kärber G. Beitrag zur kollektiven Behandlung pharmakologischer Reihenversuche. Archiv f experiment Pathol u Pharmakol. 1931; 162:480-483 (Contribution to the collective treatment of serial pharmacological trials. Archive for Pathol and Pharmac experiment).

Vaccine suspension PIC, target titer 6.4 log 10 DICC50/ml, dilution 1/200 from initial suspension comprised two steps. Step 1 (dilution to 1/10): melt 1 ml of initial suspension with 9 ml of SRL (PIC0 suspension). Step 2 (dilution to 1/20): melt 4 ml of the suspension obtained at step 1 with 76 ml of SRL (PIC1 suspension).

In the final suspension, obtained 80 ml of PIC suspension at 6.4 log 10; leaving 31 ml of PIC 6. 4 log 10 in a bottle: solution B1. Removed 1 ml of PIC at 6.4 log 10 (keep at −70° C.). Left the volume in another bottle: solution A1.

Preparation of final vaccine suspensions. Dogs in each group were vaccinated immediately after the corresponding vaccine suspension is prepared. At each step, suspensions were homogenized by rollover of the tubes. All the steps were rebuilt in the frozen ice and PSM.

Vaccine suspension A2 (group A): 64 vials of vaccine Bb CW with 0.4 ml of Al solution each were pooled with the 64 bottles and homogenizing the suspension obtained: vaccine suspension A2. containing ≈25 ml of Bb at 8.5 log 10/ml and PIC at 6.4 log 10/ml. Removed 1 ml of suspension A2: (keep at −70° C.). Vaccinated Group A dogs with 3.3 ml/A2 suspension/dog.

Vaccine suspension B2 (Group B): 2 vials of vaccine Bb BTS with 1 ml of SRL each were pooled and homogenized. Diluted 1/2 of the initial suspension: put 1 ml obtained in the previous step with 1 ml of SRL: B0 suspension. Diluted 1/32: put 1 ml of suspension B0 in 31 ml of solution B1: Vaccine suspension B2 contains 32 ml of Bb at 8.5 log 10/ml and PIC at 6.4 log 10/ml. Removed 1 ml of suspension B2: (keep at −70° C.). Vaccinated Group B dogs with 3.3 ml/B2 suspension/dog.

Dogs were randomized into different chambers and sessions of nebulization so each chamber contained approximately equal proportion of animal from each treatment group.

TABLE 12
Status of dogs before challenge

| Group | Id | D-1 | D1 | D2 | D7 | T-1 |
|---|---|---|---|---|---|---|
| A | 73316 | NEG | <LOC | <LOC | NEG | NEG |
| | 37260 | NEG | <LOC | <LOC | NEG | NEG |
| | 37270 | NEG | <LOC | <LOC | NEG | NEG |
| | 21014 | NEG | NEG | NEG | NEG | NEG |
| | 21311 | NEG | NEG | NEG | <LOC | NEG |
| | 21606 | NEG | <LOC | NEG | <LOC | NEG |
| B | 35829 | NEG | NEG | <LOC | NEG | NEG |
| | 73074 | NEG | <LOC | NEG | NEG | NEG |
| | 73127 | NEG | <LOC | NEG | NEG | NEG |
| | 20875 | NEG | <LOC | NEG | NEG | NEG |
| | 21076 | NEG | <LOC | NEG | NEG | NEG |
| | 35876 | NEG | NEG | NEG | NEG | NEG |
| C | 35873 | NEG | NEG | NEG | NEG | NEG |
| | 72619 | NEG | NEG | NEG | NEG | NEG |
| | 36084 | NEG | NEG | NEG | NEG | NEG |
| | 37128 | NEG | NEG | NEG | NEG | NEG |
| | 20817 | NEG | NEG | NEG | NEG | NEG |
| | 51149 | NEG | NEG | NEG | NEG | NEG |

Results on Clinical Scores of the Bivalent Vaccine in TSB-NA and CW Medium.

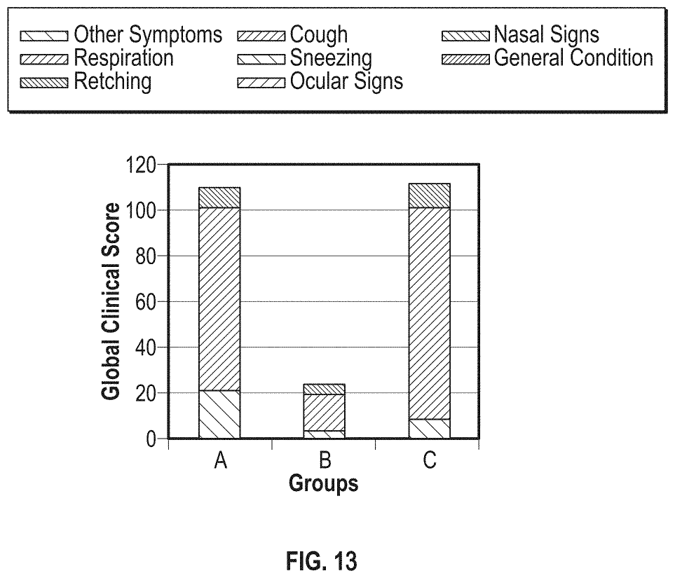

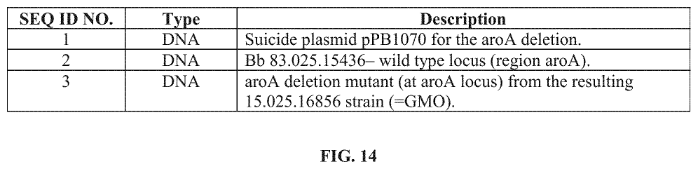

FIG. 13 shows results on clinical scores for B. bronchiseptica ΔaroA cultured in different media. The challenge was validated in the controls. Bb TSB vaccine induced a great reduction of clinical signs as compared to controls. Bb CW did not reduce clinical signs as compared to controls.

TABLE 13
Results on dogs for B. bronchiseptica ΔaroA cultured in
different media

| | | | | mean |
|---|---|---|---|---|
| | Nb of | % of | Nb of | number |
| | Dogs | Dogs | Dogs with | of days |
| | with | with | spontaneous | with |
| Gp | disease | disease | cough | cough |
| A | 6 | 100 | 6 | 5.7 |
| Bb CW | | | | |
| B | 2 | 33 | 3 | 1.4 |
| Bb STB | | | | |
| C | 6 | 100 | 6 | 7.3 |
| control | | | | |

This study shows that the Bb mutated aroA is protecting dogs by the oral route when administered in a bivalent setting (i.e., in combination with canine parainfluenza virus). What's more, in this study the group B vaccine contains Bb aroA cultured in TSB-NA medium which is free of any substance of animal origin. Thus, this vaccine provides broader protection and less risk of contamination by adventitious agents.

Group A produced in a CW medium was not protective, showing that it is unpredictable whether a selected production medium will preserve the ability of the vaccine to protect against a B. bronchiseptica challenge. Clinical protection against challenge was confirmed for vaccine Group B by Global Clinical Scores (Table 13).

Summary. The orally-delivered bivalent vaccine containing an effective amount of the disclosed attenuated B. bronchiseptica strain in combination with canine parainfluenza virus and produced in TSB-NA medium was able to elicit broad protection. The use of a non-animal product decreases the risk of contamination by adventitious agents.

### Example 6—Culture of Bordetella bronchiseptica ΔaroA in Non-Animal Origin Tryptic Soy Broth (TSB-NA)

Objectives. Culture the strongly hemolytic B. bronchiseptica ΔaroA in non-animal origin Tryptic Soy Broth.

The culture of Bordetella bronchiseptica ΔaroA was grown in filtered Tryptic Soy Broth medium non-animal origin (TSB-NA, can be purchased from Acumedia.com) and then blended 70/30% (v/v) with stabilizer.

Preparation of Medium. Prepared non-animal origin TSB (TSB-NA) according to manufacturer's directions.

Seed Flask. Prepared TSB-NA according to manufacturer's directions and filter sterilized through a 0.2 μm pore size filter. Dispensed 1000 mL of sterile TSB-NA into a sterile, 3 L disposable Erlenmeyer flask with vented cap. Held the medium for a minimum of 12 h at 37° C. to verify sterility. Immediately prior to inoculation, aseptically added 10 mL of filter sterilized 100×C AroMix to seed flask using a sterile pipette. Inoculated the flask with 1 mL from a thawed X+3 vial. Incubated the flask at 37° C. on a shaker at 200 RPM for 12 to 18 h. When the seed culture OD₆₀₀ was at 2.5±1.0, inoculated the fermentor. Seeding density was normalized to 2.75% v/v at seed culture OD₆₀₀=1. The inoculum volume was calculated using the following equation: 110/OD₆₀₀ Seed Flask=volume of inoculum (mL). Transferred appropriate volume of seed culture to sterile bottle with dip tube assembly and inoculated fermentor using a peristaltic pump.

Production Fermentation. Prepared a 7 L fermentor and sterilize for a minimum of 30 min in autoclave. Prepared and filter sterilized 4.0 L of TSB-NA medium into the sterile fermentor. Incubated the fermentor containing medium at 37° C., airflow of 0.5 vvm (2 SLPM), and agitation at 200 RPM for at least 12 h. When temperature set point was reached and stable, pH was checked using external pH meter and adjusted to process set point. Performed a zero calibration on the D0 probe using electronic zero (unplug cable), then spanned at 100% with aeration at 2 SLPM and agitation at 200 RPM. Immediately prior to inoculation, aseptically added 40 mL of sterile 100×C Aromix. Inoculated vessel with seed culture as described in section 3.2.1.2. Cultured at 37° C. for approximately 24 h EFT. Harvested culture when 700±50 mL of 30% yeast extract feed was delivered.

Culture conditions and fermentation control production fermentor. Working volume: 4 L. Temperature: 37° C. +/−0.2. pH: 7.2+/−0.1 (maintained by using 400 mL of 5 N lactic acid in a Pyrex bottle/tubing apparatus). Agitation: 200 RPM-600 RPM adjusted automatically to maintain DO set-point. Aeration: Zero initial then at 0.5 vvm (2 SLPM) constant when culture DO=30%. Pressure: n/a. Dissolved oxygen (DO): 40% maintained with constant aeration, adjustable agitation, and pure 02 supplementation. BioXpert Program: aroA_Feed_4L (Applikon Biotechnology). Feed: Feeding of 30% Bacto yeast extract started at 9 h EFT at a rate of 50 mL/h initiated by the recipe.

End of Fermentation. The end of fermentation was reached at approximately 24 h of fermentation time when 700±50 mL of yeast extract feed was delivered. Sampled the fermentor using a vacutainer. Tested for OD₆₀₀. Harvested ˜800 mL cell culture broth into a sterile 1 L bottle. Blended 420 mL of cell culture broth 70/30% (v/v) with stabilizer for a final vaccine volume of 600 mL. Blended cultures were stored at 4° C., with gentle mixing, for up to 3 days after harvest prior to lyophilization. Performed CFU testing on blended cultures after hold, prior to lyophilization. Post lyophilization samples were tested for purity, CFU, and hemolytic activity.

Process Monitoring. Implemented the BioXpert software to log online data. BioXpert Recipe: Bordetella_aroA. Recorded time course process data including sample time, pH, temperature, dissolved oxygen, impeller speed throughout the fermentation using the batch record.

Lyophilization. At harvest 420 mL of cell culture broth from the TSB-NA culture was formulated with the previously defined peptone sucrose stabilizer (Table 14) and stored for up to 3 days while mixing at 4° C. Target filling time was 24 hours after formulation. The formulation was made as shown in Table 14.

TABLE 14
Lyophilization Formulation

| | Concentration | |
|---|---|---|
| Ingredient | (w/v) | Target/dose |
| B. bronchiseptica ΔaroA | n/a | 70% v/v |
| Culture | | active |
| | | ingredient |
| Sucrose | 30% | 15% v/v |
| Dextran-Peptone | 13.3% soy peptone | 15% v/v |
| stabilizer component | 13.3% Dextran 70 | |
| | 3.33% MSG | |

The vaccines were filled at 1.1 mL per vial while mixing the blended culture. Approximately 500 3cc vials of blend were filled and loaded into the GEA lyophilizer. Testing of pre-lyophilization was done by removing 10 vials from the trays during loading to test pre lyophilization CFUs (5 vials pooled), pH, density, osmolarity, and Tg. The vaccines were lyophilized using the cycle developed for oral B. bronchiseptica AFQ2 strain as shown in Table 15.

TABLE 15
Lyophilization Cycles Steps, Temperature and Pressure

| Step | Time (min) | Temperature | Pressure |
|---|---|---|---|
| Loading | NA | 4 ° C. | ambient |
| Freezing | 80 | −45 ° C. | ambient |
| Freezing | 90 | −45 ° C. | ambient |
| Primary Drying | 105 | −10 ° C. | 80 mTorr |
| Primary Drying | 1080 | −10 ° C. | 80 mTorr |
| Secondary Drying | 131 | 28 ° C. | 80 mTorr |
| Secondary Drying | 540 | 28 ° C. | 80 mTorr |
| Stoppering Temperature | 1 | 28 ° C. | 80 mTorr |
| Nitrogen Backfill | n/a | 28 ° C. | 2.9 psia |
| | | | (150 Torr) |
| Unloading | n/a | 4 ° C. | ambient |

After lyophilization the vials were labeled, capped, and tested as outlined in Table 16.

Sampling. Table 16 shows the sampling process for the B. bronchiseptica ΔaroA fermentation batches. The fermentor was sampled before inoculation, during specific times of the fermentation process, and at the end of fermentation (HV). Performed CFUs on the HV sample. Performed microscopic checks and streak plating periodically to check sterility and observe the microorganism's morphology. After harvest, the pre-lyophilization and post-lyophilization samples were tested for CFUs.

TABLE 16
Sampling process for B. bronchiseptica ΔaroA batches

| Stage | Sample Time point | OD₆₀₀ | Purity | CFU | H+ % | DMO | pH | Dens | Osmo | Tg |
|---|---|---|---|---|---|---|---|---|---|---|
| Test Lab | | BPD | BPD | BPD | BPD | BPD | BPD | BPD | BPD | BPD |
| Quantity | | 1 mL | TSA | 1 mL | 1 mL | 0.1 mL | 1 mL | 3 mL | 1 mL | 1 mL |
| Vial | At inoculation | | ✓ | | | | | | | |
| Seed | Final | ✓ | ✓ | | | ✓ | | | | |
| Flask | | | | | | | | | | |
| Vessel | Pre-Inoculation | | ✓ | | | | | | | |
| | Culture growth | ✓ | | | | | | | | |
| | HV | ✓ | ✓ | ✓ | | ✓ | | | | |
| | PBS suspension | | | ✓ | | | | | | |
| Lyo. | Pre-lyo | | | ✓ | | | ✓ | ✓ | ✓ | ✓ |
| | Post-lyo (active | | ✓ | ✓ | ✓ | | | | | |
| | ingredient) | | | | | | | | | |
| Storage | | n/a | 37° C. | 37° C. | 37° C. | n/a | 4° C. | 4° C. | 4° C. | 4° C. |

Assessment Criteria/Data Analysis/Statistics. Interpretation of results was based on data analysis. CFU yields greater than 10′ CFU/mL for the TSB-NA culture were harvested. Lyophilization loss was calculated and was less than 0.5 log₁₀ from pre-lyophilization to post-lyophilization.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. An attenuated aroA mutant B. bronchiseptica strain capable of eliciting protective immunity against B. bronchiseptica infection in an animal when administered orally to the animal.

2. The attenuated aroA mutant B. bronchiseptica strain of claim 1, wherein the attenuated aroA mutant B. bronchiseptica strain has a partially deleted aroA gene.

3. The attenuated aroA mutant B. bronchiseptica strain of claim 1, wherein the attenuated aroA mutant B. bronchiseptica strain has a complete deletion of its aroA gene.

4. The attenuated aroA mutant B. bronchiseptica strain of claim 1, wherein the attenuated aroA mutant B. bronchiseptica strain comprises a polynucleotide having at least 85% sequence identity to SEQ ID NO:3.

5. The attenuated aroA mutant B. bronchiseptica strain of claim 1, wherein the attenuated aroA mutant B. bronchiseptica strain is deposited under the CNCM Deposit No. 1-5391.

6. An immunogenic composition comprising an attenuated aroA mutant B. bronchiseptica strain capable of eliciting an immune response when administered orally to an animal.

7. The immunogenic composition of claim 6, wherein the immunogenic composition further comprises a pharmaceutically or veterinarily acceptable carrier, adjuvant, vehicle, and/or excipient.

8. The immunogenic composition of claim 6, wherein the immunogenic composition is adjuvant-free.

9. The immunogenic composition of claim 6, wherein the immunogenic composition is a single dose formulation for oral administration.

10. The immunogenic composition of claim 9, wherein the single dose formulation has between 1×10³ CFU to 1×10¹⁰ CFU of the attenuated aroA mutant B. bronchiseptica strain.

11. The immunogenic composition of claim 9, wherein the single dose formulation has between 1×10⁸ CFU to 1×10¹⁰ CFU of the attenuated aroA mutant B. bronchiseptica strain.

12. The immunogenic composition of claim 6, further comprising a canine parainfluenza virus antigen.

13. The immunogenic composition of claim 6, further comprising a canine adenovirus antigen.

14. The immunogenic composition of claim 6, wherein the immunogenic composition is free of substances of animal origin.

15. The immunogenic composition of claim 6, wherein the immunogenic composition is a vaccine.

16. A method for eliciting a protective immune response against B. bronchiseptica in an animal, comprising:
administering to the animal an oral vaccine comprising an effective amount of an aroA mutant Bordetella bronchiseptica bacteria strain.

17. The method of claim 16, wherein the animal is a canine or a feline.

18. The method of claim 16, the protective immune response is effective to provide the animal with protection against virulent B. bronchiseptica infection, clincical disease associated with virulent B. bronchiseptica infection, and/or clinical symptoms associated with virulent B. bronchiseptica infection.

19. The method of claim 16, wherein a prime-boost administration regimen is employed.

20. The method of claim 19, wherein the animal is between 0 to 6 months old.


SEQUENCE LISTING (3 sequences)

SEQ ID NO: 1 - DNA, 5812 bp, Artificial Sequence
  pPB1070 suicide plasmid
tcgacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtcctctagatctcctctccgcgggctctgatatccttgaattccttggccggcccctccgcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggatagcccgcgcgcgattccggattaaggggacaggatgttgcaacttaccaacaatggggcgggaaaccggcttttttctgagcctgaccatagccagtcctgccgatttttgatgcaatagcgtcaacttcccttcgcggagttaggggggcggcaggccggtatgtgctgcattgctgctcttgtcactcaaatcaacggagatttcttaaatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgagcatgcaagctttcgcgagctcatttaaatcatcgcgatgacgcgaacgtggcccgcgcactggcggcattgcaggcgcaggtggccttcttcaaggtgctgggttcgtatccggcgcaatagggccacggcgtggttgatgcacaccaggcggccgccgtgacggagcgtgtgtccgtccggcgctgccatattgtttttctgcaggtatgagcgcgaacaattcggcaaacgggcaggggcccctggtccccgtgctggctgtggccggcgtaggcttgatcggtggctcgttcgccgccgcgttgcgccacgccgggcaggtcggcaccattctcggcgtgggccgcaacccggcgtcgctggcgcgtgcgcgcgagctgggcctgatcgacgaggccgtctcgcccgaggaagcggcggcgcgcgcagacctggtgctgctgtccaccccggtgggcgggctgggcgcgatgctggcgcgcatgcgcgaccatctgcggccgggctgcctgctgaccgatgccggcagcaccaaatcgcaggtcgtcatggcggcgcgccaggcgctgggcgagcaggtttcctgtttcgtgccggggcatccgatcgccggaggcgagcgcaccgggcccgaggcggccgacgcggggctgtacgtacggcgtgcggtagtgcttacgcccctgccggagaacgcggccgcatcggtggcgcgcgtgcgcgcctgctggcacgcatgcggggcgcacgtggtcgagatggacgacgtggcgcacgaccggctgctggcttcggtcagccacatgccgcacttcctggccgccgtctacatggcccaggtggccggctccgatgacgcccaggcgcgcatggatctggccggtagcggttttcgggatttcacgcgcatcgcggccggttcgccggaaatgtggcgcgatatctttttgtccaaccaggccgccatgcagtccgagctggcggccctgcgacgggtgctcgacgaggccgagcaggcattgggcgccggcgacggcgcgggcctgcaggccttgctggagcgcgcggcgcacgcgcggcgcaattggcgcaaggattccaaatgactaggactagcctagatggctatttccgcatccgccggcgccgcgccggtcatcaccatcgacggccccacggcttccggcaagggcaccatcgcgcaccgggtggccaagcagctgggctgggacgtgctcgacagcggcgccctgtaccggctgaccgccctggccgcgttgcggcgcggcctgccggccaccgacgaaccggcggtggccgccgtggcccaggcgctggacgtacggttcgacgggccgcatgtctacctggaagggcgggatgccgggcacgaaatccgccaggaagaggtcggaaactacgcttcgcgcatcgccgcctacccgggcgttcgtcaggccttgctggagcgccagcgggctttccggcagccgccgggcctggtcgccgacggccgcgacatggggacggtcgtgtttcctgatgcgtccctgaaaatatttctggtggccgatgtcgaggcgcgcgcgcaaagacgctgtaagcagttgatcgaaaagggaatttctgctaatctagatgacctgctacgcgatatgcgtgaacgcgatgcgcgcgacacgcagcgtgcggtggcaccgcttgccccggctgccgatgcgcatgtgctggattcttccggcctgaccatcgaacagaccgtacaggccgtgctggatttctggcgcgcctagccacggcgcgtgttgtttgggcagtacctcagtacctgtttgaggccctgacggccaatctccagcccccacgggacaccccgcaagggccggcgaagccggcaggttttaccactccactgctgcgggcaatccgttgcggtgtgttttgaacggccaatcccggccaaatggattccaacctaatgtcttccgtttccacctccgccatcgttggcggcgaaaacttcgccgacctgttcgcagaaagcctcaagagccaggacatgaagtccggcgaggtcatcagtgccgaagtcgtgcgcgtcgatcacaatttcgtggtcgtcaacgccggcctgaagtccgaagcgctgattccg

SEQ ID NO: 2 - DNA, 6279 bp, Artificial Sequence
  aroA locus in wild-type Bordetella bronchiseptica strain
ggtcgaacgctgaaaacaacgaaacgggagacggagatggcaagggacaagatcgtggccaggaacgtgagcatgcggttcggcgacgtactggcgatcaaggatgcgacctgcagcatcggggaacgggagtttgtcgcaatcatcggccccagcggctgcggcaaatccacctttctgtatgtgatcgcaggtttcgaagggatttcaggcggcgccgtcgaactggacggcgaaccgatacgcggcccgggtccggatcgcggcatcgtctttcaggagttcgtcctgtacccctggcgcaccgtcatggagaacgtcacgctgggcctggacatcaagaagacgccgccgcaggaagcgcggcggcgcgcccagaagtggatcactctggcggggctggacggtttcgaggatgcgtatccgtccacgctgtccggaggcatgaagcaacgggtggcgatcgcccgggcgctgacctacgacccggaggtgatcctgctggacgaaccattcggcgcgctggatgtgcagaccaagaactacatgatccatgacctgcagagcctctggacagaggcgaacaagaccatcgtcatggtgacgcacagtgtgtcggaggcggtgctgctggccgaccgcgtgctgatattcagcgcgcggccgtcctccatcattgccgacatcaagatcgacctgccccatcccaggggcgccggcgacgagggcgtgaaggcgtacgaggacgaggtgacgcgcatcctgtcggccgaagtcgacaaatccatgagcatggagcggcgcgtggcggcgtgacgcgcggcgggccggctatgccggccgcattccatattggcgcgcccgatttaaagtagtagcattagatactcctaatttgcagtcaaccataccgaggtgagagaaatgagcatgaacgaggaaaccgccgccaaactccagggcctgacccgccgtttcgccaaactggtgcacgacctgaagttcgaggacttgcccgaggaggtgctgcacaaggccaagctgatcattcgcgatggactgggcaaccaggtcgcggcttcggccatcagcgaaccggcgcggcgcgtggtcgagatcgtgcgcgaagatcctggccgtgcccaaccgcgccggcgccgtctatgacatgctggcgccgatggcggccaacggcgtttccatgacgcgcttcgaatcgcgcccggcgcgcacgggccagtgggaatactatttctacgtcgacgtgctgggccatcgcgatgacgcgaacgtggcccgcgcactggcggcattgcaggcgcaggtggccttcttcaaggtgctgggttcgtatccggcgcaatagggccacggcgtggttgacgcacaccaggcggccgccgtgacggagcgtgtgtccgtccggcgctgccatattgtttttctgcaggtatgagcgcgaacaattcggcaaacgggcaggggcccctggtccccgtgctggctgtggccggcgtaggcttgatcggtggctcgttcgccgccgcgttgcgccacgccgggcaggtcggcaccattctcggcgtgggccgcaacccggcctcgctggcgcgtgcgcgcgagctgggcctgatcgacgaggccgtctcgcccgaggaagcggcggcgcgcgcagacctggtgctgctgtccaccccggtgggcgggctgggcgcgatgctggcgcgcatgcgcgaccatctgcggccgggctgcctgctgaccgatgccggcagcaccaaatcgcaggtcgtcatggcggcgcgccaggcgctgggcgagcaggtttcctgtttcgtgccggggcatccgatcgccggaggcgagcgcaccgggcccgaggcggccgacgcggggctgtacgtacggcgtgcggtagtgcttacgcccctgccggagaacgcggccgcatcggtggcgcgcgtgcgcgcctgctggcacgcatgcggggcgcacgtggtcgagatggacgacgtggcgcacgaccggctgctggcttcggtcagccacatgccgcacttcctggccgccgtctacatggcccaggtggccggctccgatgacgcccaggcgcgcatggatctggccggtagcggttttcgggatttcacgcgcatcgcggccggttcgccggaaatgtggcgcgatatctttttgtccaaccaggccgccatgcagtccgagctggcggccctgcgacgggtgctcgacgaggccgagcaggcattgggcgccggcgacggcgcgggcctgcaggccttgctggagcgcgcggcgcacgcgcggcgcaattggcgcaaggattccaaatgagcggattggcatatctcgacctgcccgcggcgcgcctggcgcgcggcgaggtggccctgccgggttccaagagcatctccaacagggtattgctgctggccgcgctggccgaaggcagcaccgaaatcacgggcctgctcgattccgatgacacccgcgtcatgctggccgcgttgcgccagctgggcgtatcggtgggcgaggtggccgatggccgcgtgaccatcgaaggcgtggcgcgctttccgaccgaacaggccgagctgttcctaggcaacgccggcaccgcgttccggccgctgaccgcggcgctggcgttgatgggcggcgattaccgcctgtccggcgtgccgcgcatgcacgagcggcccatcggcgacctggtcgacgccttgcgccagttcggcgccgggatcgaatatctggggcaggcggggtatccgccgctgcgcatcggcggcggcagcattcgcgtcgacgggccggtgcgggtggagggctcggtgtccagccagttcctgaccgccttgctgatggccgcccccgtgctggctcggcgcagcggccaggacatcaccatcgaggtggtgggcgagctgatttccaaaccctatatcgagatcacgctcaatctgatggcgcgttttggcgtgtcggtgcggcgcgacggctggcgcgccttcacgatcgcgcgcgatgcggcctaccgcggcccgggccgcatggcgatcgagggcgatgcctcgacggcgtcgtacttcctggccctgggcgccatcggcggcgggccggtgcgcgtcaccggcgtgggcgaggacagcatccagggcgacgtggcgttcgccgcgacgctggcggcgatgggcgccgacgtgcgctatggcccgggctggatcgagacgcgcggcgtgcgggtggccgagggcggacgcctgaaggcgttcgacgccgacttcaacctgattcccgacgccgccatgacggccgcgacgctggcgctgtacgccgacggcccatgccgcctgcgcaacatcggcagctggcgcgtcaaggagaccgaccgcatccacgccatgcacaccgagctggagaagctgggggcgggcgtgcaaagcggggcggactggctggaggtggcgccgccggcgcccggcggctggcgcgacgcccacatcggcacctgggacgaccaccgcatggccatgtgcttctcgctggccgcgttcggtccggctgcggtgcgcatcctggatccgggttgcgtcagcaagactttccccgattatttcgacgtgtacgcgggcctgctggccgcgcgggactgaacggcgcctgccccatggctatttccgcatccgccggcgccgcgccggtcatcaccatcgacggccccacggcttccggcaagggcaccatcgcgcaccgggtggccaagcagctgggctgggacgtgctcgacagcggcgccctgtaccggctgaccgccctggccgcgttgcggcgcggcctgccggccaccgacgaaccggcggtggccgccgtggcccaggcgctggacgtacggttcgacgggccgcatgtctacctggaagggcgggatgccgggcacgaaatccgccaggaagaggtcggaaactacgcttcgcgcatcgccgcctacccgggcgttcgtcaggccttgctggagcgccagcgggctttccggcagccgccgggcctggtcgccgacggccgcgacatggggacggtcgtgtttcctgatgcgtccctgaaaatatttctggtggccgatgtcgaggcgcgcgcgcaaagacgctgtaagcagttgatcgaaaagggaatttctgctaatctagatgacctgctacgcgatatgcgtgaacgcgatgcgcgcgacacgcagcgtgcggtggcaccgcttgccccggctgccgatgcgcatgtgctggattcttccggcctgaccatcgaacagaccgtacaggccgtgctggatttctggcgcgcctagccacggcgcgtgttgtttgggcagtacctcagtacctgtttgaggccctgacggccaatctccagcccccacgggacaccccgcaagggccggcgaagccggcaggttttaccactccactgctgcgggcaatccgttgcggtgtgttttgaacggccaatcccggccaaatggattccaacctaatgtcttccgtttccacctccgccatcgttggcggcgaaaacttcgccgacctgttcgcagaaagcctcaagagccaggacatgaagtccggcgaggtcatcagtgccgaagtcgtgcgcgtcgatcacaatttcgtggtcgtcaacgccggcctgaagtccgaagcgctgattcccctggaagagttcctcaatgaccagggcgaactcgaagttcaacccggcgacttcgtctcggtggcgatcgattcgctggagaacggctacggcgacaccatcctgtcgcgcgaccgcgccaagcgtctgtcggcctggctgcaactggagcaggccctcgagaacggcgagctggtcaccggcacgatcaccggcaaggtcaagggcggcctgaccgtcatgaccaacggcatccgcgcgttcctgcccggttcgctggtcgacctgcgcccggtcaaggacaccacgccgtacgaaggcaagaccctcgaattcaaggtcatcaagctggaccgcaagcgcaacaacgtcgtgctgtcgcgccgccaggtgctggaagccagcatgggcgaagagcgccagaagctgctcgagacgctgcacgaaggcgcggtggtcaagggcgtggtcaagaacatcaccgactacggcgcgttcgtcgacctgggcggcatcgatggcctgctgcacatcaccgacatggcctggcgccgcgtgcgtcacccgtccgaagtcctgcaagtgggtcaggaagtcgaagccaaggtgctcaagttcgaccaggaaaagagccgcgtctccctgggcgtcaagcagctgggcgaagatccgtgggtgggcctggctcgccgctatccgcagggcacccgcctgttcggcaaggtcaccaacctgaccgactacggcgcgttcgtcgaagtcgaagccggcatcgaaggcctggtgcacgtgtccgaaatggactggaccaacaagaacgtcgatccgcgcaaggtcgtgaccctgggcgaagaagtcgaagtcatggtcctggaaatcgacgaagaccgtcgccgcatttcgctgggcatgaagcagtgccgccagaacccgtgggaagagttcgccaccaacttcaagcgtggtgacaaggtccgcggcgccatcaagtcgatcaccgacttcggcgtgttcgtcggcctgcccggcggcatcgacggcctggtccacctgtccgacctgtcgtggacggaatcgggcgaggaagccgtgcgcaacttcaagaagggcgacgagctggaagccgtggtgctgggcatcgataccgacaaagagcgcatctcgctgggtatcaagcagctcgaaggcgacccgttcaacaacttcgttgccacgcacgacaagggcgccgttgttccgggcaccatcaagtcggtcgagcccaagggcgccgtgatcaccctgtcggtggacgtggaaggctacctgcgcgcttccgagatctcctcgggccgcgtcgaggacgccaccaccgtgctgaaggctggcgagaacatcgaagccatgatcgtcaacatcgaccgcaaggcgcgttcgatccagctgtcgatcaaggcccgcgataacgccgagacggccgaaaccatccagcgcatgtccgaggcgagcgcttcgtcgggtacgacgaacctgggcgcgctgctcaaggccaagctggaccaacagcgcaacgacggttgacgtgaccaagtcgga

SEQ ID NO: 3 - DNA, 4675 bp, Artificial Sequence
  aroA locus in aroA deletion mutant Bordetella bronchiseptica strain
atggatgattccctgcaggacaagctgcggccgctgcgcgaccgcatcgacgccatcgacgcccagatcctcgacctgctgtcgcagcgcgcgcgcaccgcgcaggaagtgggctcggtcaagcacgccgcgcacgcggatggcccggtgctgcgccccgagcgcgaagccgaagtgatccgccgcttgcagcacagcaacccggggccgttccccaaggccgccgtggcagccgtctggaccgagatcatgtccgcctgccgcggcctggagcgcggcatgaccgtggcctatctcgggccgcagggctcgttctccgaacaggccgcgctcgagcatttcggccattcggtgcagcagctgccatgcccctcgttcgatgaggtgttccgtgccgtcgaggccgggcaggccgacgtcggcatggtgccggtggaaaactccaccgagggcgcggtcaaccgcagcctggacctgctgctgaacacgccgctgaccatcctgggcgagcgctcgctggtgatccgccactgcctgatgagccagtccggcacgatggacggcatcaagacgatctcggcgcatccccaggcgctggcacagtgccagggatggctgacgcgccattacccggacctcaatcgcgtggcggcggccagcaattccgaggctgcccgcgtggccgccgaggacccgacggtcgcggccatcgctggcgaagtcgccgcgccggcctggaacctgcgcgtcgtggccgccggcatccaggacgatccgcagaaccgcacgcgcttcctggctgtcggccatatcgagccattgagcagcggcaaggacaagaccagcctgatcctggccgtgcccaaccgcgccggcgccgtctatgacatgctggcgccgatggcggccaacggcgtttccatgacgcgcttcgaatcgcgcccggcgcgcacgggccagtgggaatactatttctacgtcgacgtgctgggccatcgcgatgacgcgaacgtggcccgcgcactggcggcattgcaggcgcaggtggccttcttcaaggtgctgggttcgtatccggcgcaatagggccacggcgtggttgacgcacaccaggcggccgccgtgacggagcgtgtgtccgtccggcgctgccatattgtttttctgcaggtatgagcgcgaacaattcggcaaacgggcaggggcccctggtccccgtgctggctgtggccggcgtaggcttgatcggtggctcgttcgccgccgcgttgcgccacgccgggcaggtcggcaccattctcggcgtgggccgcaacccggcctcgctggcgcgtgcgcgcgagctgggcctgatcgacgaggccgtctcgcccgaggaagcggcggcgcgcgcagacctggtgctgctgtccaccccggtgggcgggctgggcgcgatgctggcgcgcatgcgcgaccatctgcggccgggctgcctgctgaccgatgccggcagcaccaaatcgcaggtcgtcatggcggcgcgccaggcgctgggcgagcaggtttcctgtttcgtgccggggcatccgatcgccggaggcgagcgcaccgggcccgaggcggccgacgcggggctgtacgtacggcgtgcggtagtgcttacgcccctgccggagaacgcggccgcatcggtggcgcgcgtgcgcgcctgctggcacgcatgcggggcgcacgtggtcgagatggacgacgtggcgcacgaccggctgctggcttcggtcagccacatgccgcacttcctggccgccgtctacatggcccaggtggccggctccgatgacgcccaggcgcgcatggatctggccggtagcggttttcgggatttcacgcgcatcgcggccggttcgccggaaatgtggcgcgatatctttttgtccaaccaggccgccatgcagtccgagctggcggccctgcgacgggtgctcgacgaggccgagcaggcattgggcgccggcgacggcgcgggcctgcaggccttgctggagcgcgcggcgcacgcgcggcgcaattggcgcaaggattccaaatgactaggactagcctagatggctatttccgcatccgccggcgccgcgccggtcatcaccatcgacggccccacggcttccggcaagggcaccatcgcgcaccgggtggccaagcagctgggctgggacgtgctcgacagcggcgccctgtaccggctgaccgccctggccgcgttgcggcgcggcctgccggccaccgacgaaccggcggtggccgccgtggcccaggcgctggacgtacggttcgacgggccgcatgtctacctggaagggcgggatgccgggcacgaaatccgccaggaagaggtcggaaactacgcttcgcgcatcgccgcctacccgggcgttcgtcaggccttgctggagcgccagcgggctttccggcagccgccgggcctggtcgccgacggccgcgacatggggacggtcgtgtttcctgatgcgtccctgaaaatatttctggtggccgatgtcgaggcgcgcgcgcaaagacgctgtaagcagttgatcgaaaagggaatttctgctaatctagatgacctgctacgcgatatgcgtgaacgcgatgcgcgcgacacgcagcgtgcggtggcaccgcttgccccggctgccgatgcgcatgtgctggattcttccggcctgaccatcgaacagaccgtacaggccgtgctggatttctggcgcgcctagccacggcgcgtgttgtttgggcagtacctcagtacctgtttgaggccctgacggccaatctccagcccccacgggacaccccgcaagggccggcgaagccggcaggttttaccactccactgctgcgggcaatccgttgcggtgtgttttgaacggccaatcccggccaaatggattccaacctaatgtcttccgtttccacctccgccatcgttggcggcgaaaacttcgccgacctgttcgcagaaagcctcaagagccaggacatgaagtccggcgaggtcatcagtgccgaagtcgtgcgcgtcgatcacaatttcgtggtcgtcaacgccggcctgaagtccgaagcgctgattcccctggaagagttcctcaatgaccagggcgaactcgaagttcaacccggcgacttcgtctcggtggcgatcgattcgctggagaacggctacggcgacaccatcctgtcgcgcgaccgcgccaagcgtctgtcggcctggctgcaactggagcaggccctcgagaacggcgagctggtcaccggcacgatcaccggcaaggtcaagggcggcctgaccgtcatgaccaacggcatccgcgcgttcctgcccggttcgctggtcgacctgcgcccggtcaaggacaccacgccgtacgaaggcaagaccctcgaattcaaggtcatcaagctggaccgcaagcgcaacaacgtcgtgctgtcgcgccgccaggtgctggaagccagcatgggcgaagagcgccagaagctgctcgagacgctgcacgaaggcgcggtggtcaagggcgtggtcaagaacatcaccgactacggcgcgttcgtcgacctgggcggcatcgatggcctgctgcacatcaccgacatggcctggcgccgcgtgcgtcacccgtccgaagtcctgcaagtgggtcaggaagtcgaagccaaggtgctcaagttcgaccaggaaaagagccgcgtctccctgggcgtcaagcagctgggcgaagatccgtgggtgggcctggctcgccgctatccgcagggcacccgcctgttcggcaaggtcaccaacctgaccgactacggcgcgttcgtcgaagtcgaagccggcatcgaaggcctggtgcacgtgtccgaaatggactggaccaacaagaacgtcgatccgcgcaaggtcgtgaccctgggcgaagaagtcgaagtcatggtcctggaaatcgacgaagaccgtcgccgcatttcgctgggcatgaagcagtgccgccagaacccgtgggaagagttcgccaccaacttcaagcgtggtgacaaggtccgcggcgccatcaagtcgatcaccgacttcggcgtgttcgtcggcctgcccggcggcatcgacggcctggtccacctgtccgacctgtcgtggacggaatcgggcgaggaagccgtgcgcaacttcaagaagggcgacgagctggaagccgtggtgctgggcatcgataccgacaaagagcgcatctcgctgggtatcaagcagctcgaaggcgacccgttcaacaacttcgttgccacgcacgacaagggcgccgttgttccgggcaccatcaagtcggtcgagcccaagggcgccgtgatcaccctgtcggtggacgtggaaggctacctgcgcgcttccgagatctcctcgggccgcgtcgaggacgccaccaccgtgctgaaggctggcgagaacatcgaagccatgatcgtcaacatcgaccgcaaggcgcgttcgatccagctgtcgatcaaggcccgcgataacgccgagacggccgaaaccatccagcgcatgtccgaggcgagcgcttcgtcgggtacgacgaacctgggcgcgctgctcaaggccaagctggaccaacagcgcaacgacggttgacgtgaccaagtcgga